(19) United States
(12) Patent Application Publication
(10) Pub. No.: US 2025/0197804 A1
(43) Pub. Date: Jun. 19, 2025

(54) T CELLS, COMPOSITIONS COMPRISING T CELLS AND USE THEREOF

(71) Applicants: TRINCE BV, Gent (BE); UNIVERSITEIT GENT, Gent (BE)

(72) Inventors: Ranhua XIONG, Gent (BE); Jelter VAN HOECK, Gent (BE); Stijn DE MUNTER, Gent (BE); Bart VANDEKERCKHOVE, Gent (BE); Koen RAEMDONCK, Gent (BE); Stefaan DE SMEDT, Gent (BE); Kevin BRAECKMANS, Gent (BE)

(21) Appl. No.: 18/866,984

(22) PCT Filed: May 24, 2022
(86) PCT No.: PCT/EP2022/064003

§ 371 Date: Nov. 18, 2024

(51) Int. Cl. C12N 5/0783; A61K 40/11; A61K 40/31; A61K 40/42; A61P 35/00

(52) U.S. Cl. CPC C12N 5/0636; A61K 40/11; A61K 40/31; A61K 40/42; A61P 35/00; A61K 2239/31

(57) ABSTRACT

The current invention relates to a photoporated T cell, and wherein the homeostasis of said T cell after photoporation is unaffected and comparable to the homeostasis prior to said photoporation or compared to a non-photoporated T-cell. The invention further relates to a population of T cells and a pharmaceutical composition comprising a therapeutically effective amount of T cells.

### FIELD OF THE INVENTION

The present invention relates to T cells optionally comprising (macro) molecules and compositions comprising said T cells. The latter can be used in a therapeutical setting.

### BACKGROUND

Many biotechnological and biomedical applications depend on engineered cells, which requires intracellular delivery of macromolecules like DNA, RNA, peptides or proteins in vitro or ex vivo. Several cell transfection methods and techniques for producing engineered cells are known in the art but are subject to some disadvantages and problems. Engineered cells transfected by means of chemical transfection reagents or viral vectors are associated with safety concerns regarding therapeutic applications, and offer limited flexibility in terms of (macro) molecules type and size. Furthermore, engineered cells transfected by physical transfection techniques are also know. However, known physical transfection techniques suffer from an unwanted high cytotoxicity, low cell viability and/or altered homeostasis after cell transfection.

U.S. Pat. No. 10,131,876 describes a method of electroporation of T cells for subsequent therapeutic use. While electroporation has been used to introduce foreign molecules in a cell, but the technique has been hampered due to the fact that the viability of the cells after the electroporation is low, and due to the fact that the homeostasis of the cell is altered. This is a considerable drawback when it comes to producing engineered cells for therapeutic applications.

In recent years, the FDA approved several gene therapies, based on engineered T-cells. Obviously, safe engineering of these T cells with a minimal impact to their natural abilities is key. While historically, viral vectors are preferentially used to transduce T cells, they are associated with safety concerns and offer limited flexibility in terms of (macro) molecules type and size. The rapid increase of approved gene therapies and especially applications of adoptive T cell therapies, such as T cell-based cancer immunotherapy, increases the need of these safely engineered T cells. In view of the above, there remains a need in the art for further and/or improved engineered cells suitable for therapeutic use. It is an object of the current invention to provide engineered T-cells that are safe to be used in a therapeutic setting.

### SUMMARY OF THE INVENTION

The present inventors were able to provide engineered cells safe to use in a therapeutic therapy. For example, these experiments demonstrated among others successful engineered T cells suitable for cell-based cancer immunotherapy.

To this end, the present invention relates in a first aspect to a T cell according to claim 1.

The examples show that the T cell maintains a comparable homeostasis and cell proliferation after photoporation than the T cell prior to said photoporation.

Preferred embodiments of the T cell are shown in any of the claims 2 to 13.

In a second aspect, the present invention relates to a population of T cells according to claim 14.

In a third aspect, the present invention relates to a pharmaceutical composition comprising a therapeutically effective amount of T cells according to claim 15.

In a fourth aspect, the present invention relates to the T cell according to the first aspect of the invention, the population of T cells according to the second aspect of the invention or the pharmaceutical composition according to the third aspect of the invention for therapeutic use according to claim 16.

The use as described herein provides an advantageous effect in that a T cell can comprise a broad variety of molecules, resulting in a broad range of therapeutic applications. The examples show that the engineered T cells are safe and suitable for therapeutic use resolving issues known in the prior art regarding safety and regulatory concerns due to the presence of NPs in cells.

Preferred embodiments of the fourth aspect of the invention are shown in claims 17-24.

### DESCRIPTION OF FIGURES

FIG. 1: Concept of intracellular delivery by photothermal nanofibres and characterization of photothermal electrospun nanofibres. a, Schematic overview of intracellular delivery by membrane permeabilization with photothermal nanofibres. b, SEM and TEM images of electrospun PCL nanofibres containing 0 and 1 wt % IONPs. White scale bars, 2 μm (left images); 1 μm (right images). c, Histogram showing the distribution of diameters of nanofibres without IONPs, based on the analysis of 536 individual nanofibres. f, frequency. d, Diameters of nanofibres with increasing IONP content (0-5 wt %), determined from electron microscopy images (n=469, 275, 413, 536, 423 and 417; median values are given with boxes from the 25th to 75th percentiles and whiskers from the 5th to 95th percentiles). e, Confocal microscope images of nanofibres (without IONPs) shown in three dimensions, as a z projection and an exemplary horizontal section. Scale bars, 20 μm. f, The total thickness of the fibre web was measured from three-dimensional confocal z stacks with increasing electrospinning time (n=9 images obtained from three samples, the data are presented as mean±s.d.). g, The total thickness of the nanofibre webs with increasing IONP content was measured after 30 min electrospinning time (n=3 independent samples, the data are presented as mean±s.d.). h, SEM imaging at 20 kV clearly reveals IONPs within the fibres (bottom), which was not the case at a lower voltage of 1.5 kV (top). Scale bar (applies to both images), 300 nm. i, The density of IONP clusters was quantified per unit area in the SEM images (for each condition n=10 images were recorded from three PEN samples). Scale bar (applies to all three images), 2 μm. j, Schematic drawing illustrating the three parameters that were used to describe the distribution of IONPs within the nanofibres. k, The dimensionless size ^{˜I}1 quantifies the extent to which IONPs are clustered in nanofibres. The TEM images at the top illustrate three different clusterization states of IONPs embedded in nanofibres. The orange arrowheads indicate the position of the IONPs in the nanofibres. Scale bars, 200 nm. The histogram of the dimensionless size ^{˜I}1 is shown at the bottom (n=128). I, Similar images and a histogram are shown for the dimensionless parameter ^{˜I}2, which expresses the size of the clusters relative to the fibre diameter (n=128). Scale bars, 200 nm. m, Similar images and a histogram are shown for the parameter h, which is the distance between the outer surface of an IONP cluster and the surface of the fibre (n=128 clusters in three PEN samples). Scale bars, 200 nm.

FIG. 2: PEN photoporation efficiently and repeatedly delivers macromolecules to adherent and suspension cells with minimal toxicity and without potential IONPs leakage from laser-activated PEN substrates. (a) Delivery efficiency of red fluorescently labelled 10 kDa dextran (RD10) and cell viability (Calcein positive cells) were quantify as a function of laser pulse fluence for PEN webs with different amounts of IONPs: 0.02%, 0.1%, 1.0% and 2.0%. (n=3, independent experiments, mean±SD) (b) Jurkat cell viability and delivery efficiency of RD10 shown as function of laser fluence and IONPs content of 1.0%, 2.0% and 5.0%; and cell viability and delivery efficiency of FD10 in Jurkat cells for repeated PEN photoporation using 2% IONPs and I=0.08 J/cm². (n=3, independent experiments, mean±SD) (c) Schematic overview of the experimental procedure to determine the iron content in cells by ICP-MS/MS after PEN photoporation. (d) The iron concentration was measured in untreated cells (negative control), cells incubated with IONPs with or without laser scanning (positive controls), and cells treated by PEN photoporation. For Hela cells PEN substrates with 1% IONPs were used, while it was 2% for Jurkat cells. Laser fluences were varied from 0.08 to 0.16 J/cm², with repeated photoporation from N=1 to 4 times. (n=4 independent experiments, mean±SD, *P=0.015, ***P=1.972×10⁻⁴, **P=0.0018, **P=0.0014 from left to right, one-way ANOVA). (e) Schematic overview of the experimental procedure to measure potential iron leakage from laser-activated PEN substrates into DI water. (f) The iron concentration was determined by ICP-MS/MS in DI water (negative control), in aqua regia in which an amount of fibers comparable to one PEN culture well with IONPs content of 1%, 2% or 5% (positive control) were digested, and in DI water collected from the PEN substrates after laser activation. PEN substrates with 1 and 5% IONPs were tested, without and with N=1 and 4 times laser activation at a laser fluence of 0.08 and 0.16 J/cm². (n=3, independent experiments, mean±SD).

FIG. 3: PEN photoporation for siRNA gene silencing or CRISPR/Cas9 mediated gene knockout in H1299. (a) Schematic overview of the experimental procedure to deliver SIRNA or RNPs into GFP expressing H1299 cells by PEN photoporation. (b) Confocal images showing GFP expressing H1299 cells PEN photoporated with 5 μM control (left) and anti-GFP siRNA (right). PEN substrates contained 1% IONPs and were 1× scanned with a laser fluence of 0.08 J/cm². (c) The corresponding flow cytometry histograms show how GFP expression is distributed over the cell population when PEN photoporated with control or anti-GFP siRNA. (d-e) H1299 cells were on the one hand 1×PEN photoporated with increasing concentrations of siRNA (0.5, 1, 2, 5 μM), and on the other hand multiple times (N=2, 3, 4). The MFI (d) and knockdown efficiency (e) were quantified by flow cytometry (n=3, independent experiments, mean±SD). (f) The corresponding cell viability is shown as measured by the Cell Titer Glo assay. (g) Exemplary confocal images showing eGFP expression in H1299 cells before and 48 h after PEN photoporation with 4 μM RNPs targeted to the eGFP gene (I=0.08 J/cm², 1% IONPs). Scale bars are 200 μm. (h) Corresponding flow cytometry histograms. (i-j) H1299 cells were either 1×PEN photoporated (I=0.08 J/cm²) with increasing concentrations of RNPs (0.5, 1, 2, 4 μM), or multiple times (N=2, 3, 4) for a RNP concentration of 0.5 μM. The MFI (i) and knockout efficiency (j) were measured by flow cytometry (n=3, independent experiments, mean±SD).

FIG. 4: PEN photoporation enables efficient intracellular delivery of macromolecules, including CRISPR/Cas9 ribonucleoprotein complexes, in human embryonic stem cells (hESC) without affecting cell functionality. (a) Delivery efficiency, cell viability and the delivery yield were quantified 2 h after PEN photoporation as a function of laser pulse fluence (I=0.04, 0.08, 0.12 and 0.24 J/cm²) and repeated PEN photoporation (N=2, 4; I=0.08 J/cm²) (n=3, independent experiments, mean±SD). (b) Delivery efficiency, cell viability and the delivery yield for different hESC electroporation programs measured 2 h after treatment. (c) Confocal images show green fluorescence from Calcein-AM viability staining, red fluorescence from PI positive dead cells and magenta from RD10 (Scale bar 50 μm). (d) The viability and yield were measured 24 h after treatment with PEN photoporation or electroporation using the most optimal protocols (I=0.08 J/cm², N=2 for PEN photoporation and the CE-118 program for electroporation). (e) Cell proliferation post PEN photoporation and electroporation using the most optimal protocols (n=3, independent experiments, mean±SD). (f) Confocal images of hESCs immunostained for pluripotency transcription factors Oct4 (Pou5f1), Sox2 and Nanog 24 h after PEN photoporation. Nuclei are stained by Hoechst (Scale bar 50 μm). (g) Expression of Oct4 (Pou5f1), Sox2 and Nanog relative to untreated cells as quantified from confocal images (n=3, independent experiments, mean±SD). (h) Confocal images of hESCs differentiated into cardiomyocytes and immunostained for the cardiomyocyte-specific markers TNNT2 and NKX2.5. (i) Expression of TNNT2 and NKX2.5 relative to untreated cells as quantified from confocal images (Scale bar 200 μm) (n=3, independent experiments, mean±SD). (j) Sanger sequences of non-treated hESCs (NTC), hESCs 30 PEN photoporated without RNP (PEN Ctrl), hESCs PEN photoporated with 2 μM mock RNPs (RNP Ctrl) and hESCs PEN photoporated with 2 μM IL-2R RNPs (IL-2R RNP). (k) IL-2R knockout efficiency by Sanger sequencing and tracking of indels by decomposition (TIDE) analysis. All analyzed results have a model fit R²>0.9, which indicates how well the indel distribution fits the sanger sequencing data (ICE v2 analysis by Synthego) (n=3, independent experiments, mean±SD).

FIG. 5: PEN photoporation enables efficient intracellular delivery of siRNA into T cells. PEN photoporation enables efficient intracellular delivery of siRNA into human donor-derived T cells with minimal toxicity, optimal T cell fitness and retention of T cell effector functions in vitro. (a) FD10 delivery efficiency, viability and delivery yield in human T cells photoporated with hydrated or neutral PEN nanofibres. (b) Screening of different electroporation programs for optimal FD10 delivery efficiency, viability and delivery yield. (c) Exemplary histograms showing PD1 expression in CD3+ T cells 48 h after PEN photoporation of T cells with 4 μM siPD1. (d) Optimization of PD1 silencing as a function of siPD1 concentration using the optimized PEN photoporation and electroporation delivery protocols. (e) Impact on cell size 1 h after PEN photoporation and electroporation. The values are expressed relative to the NTC. (f) Calcium levels in non-treated cells compared with PEN-photoporated or electroporated T cells 1, 6 and 24 h post-treatment. The values are expressed relative to the NTC. g, The secretion levels of several key pro-inflammatory and anti-inflammatory cytokines, expressed as fold change relative to the NTC, were measured in the supernatant of human T cells 24 and 48 h after electroporation or PEN photoporation. (h) Expression levels of the activation markers CD137 and CD154, as well as the activation/exhaustion marker PD1, were measured 24 and 48 h after electroporation or PEN photoporation and compared with those of the NTC (relative fold change). (i) The proliferation of PEN-photoporated or electroporated T cells (without one or more (macro) molecules otherwise not present in a native T cell), expressed as the relative number of cells (N/N0), was measured up to 72 h after stimulation with CD3/CD28 tetrameric antibody complexes in the presence of IL-2. (j) The cytolytic activity was measured by a standard 4 h chromium-51 release assay. The statistical significance relative to the NTC is indicated when appropriate (for all plots, n=3 biologically independent samples, the data are presented as mean±s.d., one-way ANOVA).

FIG. 6: PEN photoporation also retains T cell effector functions in vivo. (a) Schematic overview of the experimental procedure to deliver siRNA into previously transduced CAR T cells by PEN photoporation to demonstrate efficacy in a SKOV3 tumour mouse model. (b) The tumour size was monitored over time in mice intravenously injected with CAR T cells (n=5 mice), CAR T cells PEN-photoporated with siPD1 (n=4 mice) or CAR T cells combined with PD1 antibody administration (positive control, n=4 mice) and compared with that of control mice treated with PBS buffer alone (n=4 mice). The dotted line indicates the average relative tumour size in mice treated with PBS. The statistical significance relative to NTC is indicated when appropriate (the data are presented as mean±s.d., one-way ANOVA).

FIG. 7: T cell viability after electroporation. (a) Human T cells were electroporated with Nucleofector programs EO-100, EO-115 and FI-115 according to the manufacture's recommendations. Viability was measured by Calcein AM (live-death stain) and Cell Titer Glo (metabolic activity) after 2 h. (n=3, biological independent samples, mean±SD) (b) An exemplary large field of view confocal image of cells collected in a well of a 96-well titer plate. NTC=negative control cells, +EP=post electroporation with program EO-115. Viable cells were stained by Calcium AM (green) while dead cells were stained with Propidium Iodide (red). N is the total number of cells (live+dead) as counted by image processing. The insets show an enlarged image of part of the wells. (c) Quantification of the percentage of cells lost after EP, both 1 h and 24 h after electroporation with program EO-115. (n=3, biological independent samples, mean±SD).

FIG. 8: siRNAs used for PD1 silencing in human donor-derived T cells.

FIG. 9: PD1 protein expression levels of human T cells 24 hours after transfection with 1 μM of various commercial PD1 siRNA constructs (n=3, biological independent samples, mean±SD).

FIG. 10: Representative flow cytometry histogram for CD70 tumor-antigen expression and PD-L1 expression on SKOV3 and H1650 cells. The values on the top left indicate the mean fluorescence intensity.

FIG. 11: (a) Tumor size are as a function of days post SKOV3 injection (n=4 for PBS, n=5 for CAR T cells, n=4 for PEN photoporated CAR T cells with siPD1, n=4 for CAR T cells with antibody administration biological independent animals, mean±SD). They are shown for individual mice injected with (b) PBS (negative control, 4 mice), (c) CAR T cells (5 mice), (d) PEN photoporated CAR T cells with siPD1 (4 mice), and (e) CAR T cells with antibody administration (positive control, 4 mice).

FIG. 12: (a) Schematic representation of the electrospinning setup with a high voltage power supply, a digital syringe pump and a grounded rotating collector. (b) Photographs of microscope glass slides with a web of nanofibers on top containing different amounts of IONPs. (c) The size of individual IONP was quantified from TEM images and shown here as a size distribution (n=103). The mean diameter was 162 nm with a standard deviation of 41 nm. The inset shows an exemplary TEM image. (d) High resolution TEM images showing that IONP are embedded close to the PCL fiber surface but are still covered by a thin layer of PCL polymer. The outer surface of the IONP and nanofibers is demarcated by white and yellow dotted lines, respectively. (e) The parameters Γ1, Γ2 and h were quantified from TEM images of nanofibers prepared with 1% and 5% IONPs (n=53). (n=53, mean±SD, one-way ANOVA).

FIG. 13: (a) Singlet human T cells were gated based on the forward scatter (FSC) and side scatter (SSC) signals. (b) The gated singlet cell population consisted of nearly 100% CD3+ T cells according to the PB450 CD3 antibody fluorescence. (c) The CD3+ T cell population included ˜45% CD4+ cells labelled by the PC5.5 CD4 antibody. (d) The rest of the population primarily consisted of ˜53% CD8+ cells labelled by the APC CD8 antibody.

FIG. 14: Intracellular delivery of FD10 in primary human T cells by PEN photoporation (neutral fibers) (a) and viability as measured by Cell Titre Glo after 2 h (b) and the delivery yield (c) were determined as a function of laser fluence and IONP content. The FD10 concentration was 2 mg/ml. (n=3, biological independent samples, mean±SD).

FIG. 15: (a) Schematic overview of the experimental procedure to deliver macromolecules into cells by PEN photoporation. (b) Photograph of microscope glass slides with nanofibers and adhesive stickers that are being sterilized by UV treatment in a laminar flow hood. (c) Schematic representation of how the homemade PEN culture wells are prepared. An eight well adhesive sticker (silicone) is applied onto a PEN web that is still attached to a glass slide. The top plastic layer is a removable protective layer on the top side of the silicone stickers. (d) After removal of the web and sticker from the glass slide, the sample is cut so as to obtain individual culture wells with PEN bottom. A photograph is shown of an individual PEN culture well held up in the air (top) or applied in a 6-well plate filled with water.

FIG. 16: (a) Schematic representation of the experimental procedure to modify the PCL fibers with collagen for better cell attachment. (b) Confocal images of Hela cells grown in a PEN cell culture well (1% IONPs) without (left) and with collagen-coating applied to the fibers (right). Cells were labeled with the calcein-AM viability stain. (c) The cell density and cell area were quantified by image processing (n=7 from independent three samples, the thick horizontal lines in the box plot indicate the median value; the boxed area extends from the 25th to 75th percentile with whiskers from the 5th to the 95th). (d) The number of IONPs clusters available per cell was calculated by multiplying the IONPs density (see FIG. 15i, n=10 images from three independent experiments, mean±SD) with the cell area for increasing percentages of embedded IONPs. (e) Red fluorescent dextran of 10 kDa (RD10) was delivered into Hela cells by PEN (1% IONPs) photoporation. Confocal images show green fluorescence from the Calcein-AM viability staining and red fluorescence from RD10. Transfection efficiency increases with increasing laser pulse fluence (25.6%±13.0% RD10 positive cells for I=0.04 J/cm2, 86.8%±6.9% RD10 positive cells for I=0.08 J/cm2, and 92.2%±10.0% RD10 positive cells for I=0.12 J/cm2). At the highest laser fluence cell toxicity becomes apparent from a loss of green cells (88.6%±10.2% viability for I=0.04 J/cm2, 87.8%±8.1% viability for I=0.08 J/cm2, and 56.9%±19.6% viability for I=0.12 J/cm2).

FIG. 17: (a) Hela cells are incubated with free IONP, allowing the nanoparticles to attach to the cell membrane. Cells are subsequently washed and irradiated with pulsed laser light in the presence of FD10. The percentage of FD10 positive cells is quantified by flow cytometry, while cell viability is assessed in parallel by the Cell Titer Glo assay. IONP concentration was gradually increased from 2.25×108 to 9.0×108 NPs/ml in combination with a laser pulse fluence ranging from 0.32 to 1.60 J/cm2. (n=3, independent experiments, mean±SD) (b) Quantification of delivery efficiency after N laser irradiation steps (N=2, 3, 4 and 5). Cells treated with IONP (4.5×108 NPs/ml) were first irradiated N−1 times, after which FD10 was added and delivered into the cells by the Nth irradiation cycle. This shows the remaining photoporation efficiency upon the Nth photoporation cycle. (n=3, independent experiments, mean±SD) (c) IONP morphology after irradiation with N laser pulses of 1.26 J/cm2. N=0 corresponds to unirradiated IONP. Scale bars are 500 nm. (d) The number of VNBs within the laser irradiation zone was counted for increasing laser fluence using free IONPs suspended in water. The solid line is a Boltzmann fit, from which the VNB threshold fluence can be derived (Ith=1.07 J/cm²), defined as the laser fluence at which 90% of the particles generate VNBs.

FIG. 18: (a) Repeated photoporation with PEN webs is demonstrated by sequentially delivering RD10 (red fluorescence) and FD10 (green fluorescence). The overlay shows that many cells have both green and red fluorescence. (b) Flow cytometry data showing red (RD10) fluorescence in the x-axis and green (FD10) fluorescence in the y-axis. Approximately 90% of the cells are positive for both red and green fluorescence after repeated photoporation. (c-d) Repeated photoporation of cells on PEN webs was demonstrated by FD10, the concentration of which was doubled between each scan from 0.2 to 1.6 mg/mL. The percentage of positive cells was quantified (c) along with the rMFI (relative mean fluorescence intensity) per cell (d). (e) The extent to which PEN webs loose transfection capacity upon each subsequent round of photoporation was investigated by photoporating cells N−1 times in the presence of normal cell medium (without marker) and the last time in the presence of FD10. The dashed line indicates the percentage of positive cell by one laser scan as a reference. (f) The corresponding cell viability was determined by Calcein AM. The dashed line indicates cell viability for one laser scan. (n=3 independent experiments, mean±SD)

FIG. 19: (a-b) SEM and TEM images of PEN webs after a single laser scan of increasing laser fluence (a), or after multiple laser scans at a fixed laser fluence of 0.08 J/cm2 (b). The photoporation procedure was repeated with the same PEN substrates up to 4 times (N=1, 2 and 4). (c) The effective photothermal area on the nanofiber surface was calculated for I=0.08 J/cm2 as a function of the number of IONP either as a linear arrangement of individual neighboring IONP or as an equivalent larger spherical particle of the same total volume. The effective photothermal area is defined as the average area of the nanofiber surface that reaches a temperature above 60° C.

FIG. 20: FITC-dextrans from 10 to 500 kDa were delivered into Hela cells by PEN photoporation using fibers with 1% IONPs. Uptake was determined by flow cytometry and expressed as the percentage of positive cells (a) and rMFI (b). (n=3, independent experiments, mean±SD)

FIG. 21: (a) A z-projection confocal view of Jurkat cells collected on the PEN substrate is shown in the top panel. A sliced view is presented in the bottom panel showing a focal plane where both the fibers and the bottom of the cells are visible. The plasma membrane was labelled by CellMask (red) while the nanofibers were labeled with Coumarin 6 (green). (b) Quantification of the number of IONPs clusters per Jurkat cell for increasing percentages of IONPs embedded in the nanofibers (n=10 images from three independent experiments, mean±SD). (c) FD10 was photoporated into Jurkats with a PEN substrate containing 1% IONPs (n=3, independent experiments, mean±SD, one-way ANOVA). The nanofibers were either neutral (unmodified), positively charged (PAH treated) or collagen-coated. Photoporation was each time performed once with a laser pulse fluence of 0.08 J/cm². Differences between two datasets were assessed using one-way ANOVA. Statistical significance is indicated as follows: ns P≥0.05, **P<0.00.

FIG. 22: (a) ROS generation was determined using 2′, 7′-Dichlorofluorescin (DCFH) as a fluorescent indicator. A 10 μM H2O2 solution was used as positive control. ROS generation was measured in DI water collected from PEN substrates containing different amounts of IONPs (0, 1, 5%), with and without laser irradiation at different fluences (0, 0.08, 0.16 J/cm²) (n=3, independent experiments, mean=SD, one-way ANOVA). (b) Exemplary dark field images recorded just before, during and just after the arrival of a single laser pulse on a PEN web with 1.0% IONPs. At a laser pulse fluence of 0.56 J/cm² VNBs can be seen in the middle image within the green circles. At a lower laser fluence of 0.14 J/cm², on the other hand, VNBs could not be observed. Dashed circles indicate the laser irradiation region. (c) The number of VNBs within the laser irradiation zone was counted for increasing laser fluence using PEN substrates with 0.02% and 2% IONPs (n=15 images from three independent experiments, mean±SD). The solid line is a Boltzmann fit, from which the VNBs threshold fluence can be derived, defined as the laser fluence at which 90% of the particles generate VNBs.

FIG. 23: Local transient heating is the mechanism behind cell membrane permeabilization by PEN photoporation. (a) Schematic overview of theoretical calculations on laserinduced heat generation in IONPs followed by heat transport in the fiber and surrounding cell medium. (b) The extinction spectrum of 160 nm IONPs in dispersion was measured by UV-VIS spectrometry and calculated from Mie theory. (c) The temperature was calculated for a single IONP after absorption of a 7 ns laser pulse at 647 nm for increasing laser fluences. (d) Example of a simulation on the heat transfer from a heated IONP embedded in a PCL nanofiber and surrounded by water. The IONP initial temperature was calculated from the absorption of a 7 ns 0.08 J/cm2 laser pulse. The IONPs were positioned at a distance h=40 nm from the fiber surface. The orange dashed lines indicate the boundary of the nanofiber. (e-f) Time dependency of parameters T (e) and A (f) for N=1, h=40 nm and I=0.08 J/cm2. Here, A is the total area of the fiber surface with a temperature >60° C., while T is average temperature of this area. T and Aare the time averages of T and A. (g-j) Systematic evaluation of the effect of laser fluence (I), distance between IONPs and fiber surface (h) and number of clustered IONPs (N=1 (h), N=2 (i), N=3 (j), N=4 (k)) on T and Avia numerical simulation.

FIG. 24: (a) The IONPs absorption cross-section spectrum was calculated from the generalized multiparticle Mie theory for different linear particle arrangements (N=1, 2, 4 and 8 IONPs separated by 1 nm interparticle distance). The inserted graph shows the absorption cross-section at the wavelength of 647 nm for the various linear IONPs arrangements. (b) The absorption cross-section is shown, calculated from Mie theory, of a single IONPs in water, PCL polymer and a 50% water-50% polymer effective medium. (c) The overall 3D geometry of the model used for numerical simulations is shown (left) with a zoom-in of the central region that contains the IONPs embedded in the central fiber (right). (d) The simulation domain is discretized into a grid which is shown for the entire simulation space on the left, and for the central nanoparticle region on the right.

FIG. 25: FD500 delivery in Hela cells by PEN photoporation with a 1% IONPs substrate for increasing laser fluences. The percentage of positive cells and the rMFI was determined by flow cytometry (n=3, independent experiments, mean±SD)

FIG. 26: The ratio of the fiber surface area that reaches a temperature above the spinodal temperature As vs. the area that reaches a temperature above 60° C. Ae is plotted as a function of depth h of the IONP cluster below the fiber surface. Calculations were performed for clusters of N=1, 2, 4 and 8 IONP irradiated with a single laser pulse of (a) 0.08 J/cm2, and (b) 0.16 J/cm2.

### DETAILED DESCRIPTION OF THE INVENTION

The present invention relates to a T cell, a population of T cells and a pharmaceutical composition comprising an effective therapeutic amount of T cells.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

The term “in vitro” as used herein is to denote outside, or external to, animal or human body. The term “in vitro” as used herein should be understood to include “ex vivo”.

The term “ex vivo” typically refers to tissues or cells removed from an animal or human body and maintained or propagated outside the body, e.g. in a culture vessel.

The term “in vivo” as used herein is to denote inside, or internal to, animal or human body.

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

The term “nucleic acid” as used herein typically refers to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Exemplary modified nucleobases include without limitation 5-substituted pyrimidines, 6-azapyrimidines and N-2, N-6 and O-6 substituted purines, including 2-aminopropyladenine, 5-propynyluracil and 5-propynylcytosine. In particular, 5-methylcytosine substitutions have been shown to increase nucleic acid duplex stability and may be preferred base substitutions in for example antisense agents, even more particularly when combined with 2′-O-methoxyethyl sugar modifications. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups (such as without limitation 2′-O-alkylated, e.g., 2′-O-methylated or 2′-O-ethylated sugars such as ribose; 2′-O-alkyloxyalkylated, e.g., 2′-O-methoxyethylated sugars such as ribose; or 2′-O,4′-C-alkylene-linked, e.g., 2′-O,4′-C-methylene-linked or 2′-O,4′-C-ethylene-linked sugars such as ribose; 2′-fluoro-arabinose, etc.). Nucleic acid molecules comprising at least one ribonucleoside unit may be typically referred to as ribonucleic acids or RNA. Such ribonucleoside unit(s) comprise a 2′-OH moiety, wherein-H may be substituted as known in the art for ribonucleosides (e.g., by a methyl, ethyl, alkyl, or alkyloxyalkyl). Preferably, ribonucleic acids or RNA may be composed primarily of ribonucleoside units, for example, ≥80%, ≥85%, ≥90%, ≥95%, ≥96%, ≥97%, ≥98%, ≥99% or even 100% (by number) of nucleoside units constituting the nucleic acid molecule may be ribonucleoside units. Nucleic acid molecules comprising at least one deoxyribonucleoside unit may be typically referred to as deoxyribonucleic acids or DNA. Such deoxyribonucleoside unit(s) comprise 2′-H. Preferably, deoxyribonucleic acids or DNA may be composed primarily of deoxyribonucleoside units, for example, ≥80%, ≥85%, ≥90%, ≥95%, ≥96%, ≥ 97%, ≥98%, ≥99% or even 100% (by number) of nucleoside units constituting the nucleic acid molecule may be deoxyribonucleoside units. Nucleoside units may be linked to one another by any one of numerous known inter-nucleoside linkages, including inter alia phosphodiester linkages common in naturally-occurring nucleic acids, and further modified phosphate- or phosphonate-based linkages such as phosphorothioate, alkyl phosphorothioate such as methyl phosphorothioate, phosphorodithioate, alkylphosphonate such as methylphosphonate, alkylphosphonothioate, phosphotriester such as alkylphosphotriester, phosphoramidate, phosphoropiperazidate, phosphoromorpholidate, bridged phosphoramidate, bridged methylene phosphonate, bridged phosphorothioate; and further siloxane, carbonate, sulfamate, carboalkoxy, acetamidate, carbamate such as 3′-N-carbamate, morpholino, borano, thioether, 3′-thioacetal, and sulfone internucleoside linkages. Preferably, inter-nucleoside linkages may be phosphate-based linkages including modified phosphate-based linkages, such as more preferably phosphodiester, phosphorothioate or phosphorodithioate linkages or combinations thereof. The term “nucleic acid” also encompasses any other nucleobase containing polymers such as nucleic acid mimetics, including, without limitation, peptide nucleic acids (PNA), peptide nucleic acids with phosphate groups (PHONA), locked nucleic acids (LNA), morpholino phosphorodiamidate-backbone nucleic acids (PMO), cyclohexene nucleic acids (CeNA), tricyclo-DNA (tcDNA), and nucleic acids having backbone sections with alkyl linkers or amino linkers (see, e.g., Kurreck 2003 (Eur J Biochem 270:1628-1644)). “Alkyl” as used herein particularly encompasses lower hydrocarbon moieties, e.g., C1-C4 linear or branched, saturated or unsaturated hydrocarbon, such as methyl, ethyl, ethenyl, propyl, 1-propenyl, 2-propenyl, and isopropyl. Nucleic acids as intended herein may include naturally occurring nucleosides, modified nucleosides or mixtures thereof. A modified nucleoside may include a modified heterocyclic base, a modified sugar moiety, a modified inter-nucleoside linkage or a combination thereof.

The term “nucleic acid” preferably encompasses DNA, RNA and DNA/RNA hybrid molecules, specifically including hnRNA, pre-mRNA, mRNA, cDNA, genomic DNA (gDNA), plasmid DNA (pDNA), amplification products, oligonucleotides, and synthetic (e.g., chemically synthesised) DNA, RNA or DNA/RNA hybrids. RNA is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA). A nucleic acid can be naturally occurring, e.g., present in or isolated from nature, can be recombinant, i.e., produced by recombinant DNA technology, and/or can be, partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

The term “oligonucleotide” as used throughout this specification refers to a nucleic acid (including nucleic acid analogues and mimetics) oligomer or polymer as defined herein. Preferably, an oligonucleotide, such as more particularly an antisense oligonucleotide, is (substantially) single-stranded. Oligonucleotides as intended herein may have a length of about 10 to about 100 nucleoside units (i.e., nucleotides or nucleotide analogues), preferably about 15 to about 50, more preferably about 20 to about 40, also preferably about 20 to about 30 nucleoside units (i.e., nucleotides or nucleotide analogues). Oligonucleotides as intended herein may comprise one or more or all non-naturally occurring heterocyclic bases and/or one or more or all non-naturally occurring sugar groups and/or one or more or all non-naturally occurring inter-nucleoside linkages, the inclusion of which may improve properties such as, for example, increased stability in the presence of nucleases and increased hybridization affinity, increased tolerance for mismatches, etc.

Nucleic acid binding agents, such as oligonucleotide binding agents, are typically at least partly antisense to a target nucleic acid of interest. The term “antisense” generally refers to an agent (e.g., an oligonucleotide) configured to specifically anneal with (hybridize to) a given sequence in a target nucleic acid, such as for example in a target DNA, hnRNA, pre-mRNA or mRNA, and typically comprises, consist essentially of or consist of a nucleic acid sequence that is complementary or substantially complementary to said target nucleic acid sequence. Antisense agents suitable for use herein, such as hybridization probes or amplification or sequencing primers and primer pairs) may typically be capable of annealing with (hybridizing to) the respective target nucleic acid sequences at high stringency conditions, and capable of hybridizing specifically to the target under physiological conditions. The terms “complementary” or “complementarity” as used throughout this specification with reference to nucleic acids, refer to the normal binding of single-stranded nucleic acids under permissive salt (ionic strength) and temperature conditions by base pairing, preferably Watson-Crick base pairing. By means of example, complementary Watson-Crick base pairing occurs between the bases A and T, A and U or G and C. For example, the sequence 5′-A-G-U-3′ is complementary to sequence 5′-A-C-U-3′. The reference to oligonucleotides may in particular but without limitation include hybridization probes and/or amplification primers and/or sequencing primers, etc., as commonly used in nucleic acid detection technologies.

The terms “ribozyme” or “ribonucleic acid enzymes” as used herein refer to RNA molecules that have the ability to catalyse specific biochemical reactions, for example RNA splicing in gene expression. The function of ribozymes is similar to the action of protein enzymes. The most common activities of ribozymes are the cleavage or ligation of RNA and DNA and peptide bond formation. Within the ribosome, ribozymes function as part of the large subunit ribosomal RNA to link amino acids during protein synthesis. They also participate in a variety of RNA processing reactions, including RNA splicing, viral replication, and transfer RNA biosynthesis. Examples of ribozymes include the hammerhead ribozyme, the VS ribozyme, Leadzyme and the hairpin ribozyme.

The term “protein” as used herein generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native protein, such as, e.g., amino acid deletions, additions and/or substitutions. The term contemplates both full-length proteins and protein parts or fragments, e.g., naturally occurring protein parts that ensue from processing of such full-length proteins.

The term “polypeptide” as used herein encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally occurring polypeptide parts that ensue from processing of such full-length polypeptides.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest, i.e., antigen-binding fragments), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256:495), or may be made by recombinant DNA methods (e.g., as in U.S. Pat. No. 4,816,567). Monoclonal antibodies may also be isolated from phage antibody libraries using techniques as described by Clackson et al. 1991 (Nature 352:624-628) and Marks et al. 1991 (J Mol Biol 222:581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments, single domain (sd) Fv, such as VH domains, VL domains and VHH domains; diabodies; linear antibodies; single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

The term “lipid” as used herein refers to a macromolecule that is soluble in a nonpolar solvent. Lipids may be divided into eight categories: fatty acids; glycerolipids; glycerophospholipids; sphingolipids; saccharolipids; polyketides; sterol lipids or sterols; and prenol lipids or prenols.

The term “gene editing system” or “genome editing system” as used herein refers to a tool to induce one or more nucleic acid modifications, such as DNA or RNA modifications, into a specific DNA or RNA sequence within a cell. Targeted genome modification is a powerful tool for genetic manipulation of cells and organisms, including mammals. Genome modification or gene editing, including insertion, deletion or replacement of DNA in the genome, can be carried out using a variety of known gene editing systems. Gene editing systems typically make use of an agent capable of inducing a nucleic acid modification. In certain embodiments, the agent capable of inducing a nucleic acid modification may be a (endo) nuclease or a variant thereof having altered or modified activity. (endo) Nucleases typically comprise programmable, sequence-specific DNA- or RNA-binding modules linked to a nonspecific DNA or RNA cleavage domain. In DNA, these nucleases create site-specific double-strand breaks at desired locations in the genome. The induced double-stranded breaks are repaired through nonhomologous end-joining or homologous recombination, resulting in targeted mutations. In certain embodiments, said (endo) nuclease may be RNA-guided. In certain embodiments, said (endo) nuclease can be engineered nuclease such as a Clustered Regularly Interspaced Short Palindromic Repeats (CRISPR) associated (Cas) (endo) nuclease, such as Cas9, Cpf1, or C2c2, a (zinc finger nuclease (ZFN), a transcription factor-like effector nuclease (TALEN), a meganuclease, or modifications thereof. Methods for using TALEN technology, Zinc Finger technology and CRISPR/Cas technology are known by the skilled person.

The term “cell” refers to all types of biological cells, including eukaryotic cells and prokaryotic cells. As used herein, the terms “cells” and “biological cells” are interchangeably used.

The terms “blood cell”, “hematopoietic cell”, “hemocyte” or “hematocyte” refer generally to a cell produced through hematopoiesis and found mainly in the blood. Major types of blood cells include red blood cells (erythrocytes), white blood cells (leukocytes), and platelets (thrombocytes).

The term “stem cell” refers generally to an unspecialized or relatively less specialized and proliferation-competent cell, which is capable of self-renewal, i.e., can proliferate without differentiation, and which or the progeny of which can give rise to at least one relatively more specialized cell type. The term encompasses stem cells capable of substantially unlimited self-renewal, i.e., wherein the progeny of a stem cell or at least part thereof substantially retains the unspecialized or relatively less specialized phenotype, the differentiation potential, and the proliferation capacity of the mother stem cell, as well as stem cells which display limited self-renewal, i.e., wherein the capacity of the progeny or part thereof for further proliferation and/or differentiation is demonstrably reduced compared to the mother cell. By means of example and not limitation, a stem cell may give rise to descendants that can differentiate along one or more lineages to produce increasingly relatively more specialized cells, wherein such descendants and/or increasingly relatively more specialized cells may themselves be stem cells as defined herein, or even to produce terminally differentiated cells, i.e., fully specialized cells, which may be post-mitotic.

The term “isolated” as used throughout this specification with reference to a particular component generally denotes that such component exists in separation from—for example, has been separated from or prepared and/or maintained in separation from—one or more other components of its natural environment. More particularly, the term “isolated” as used herein in relation to cells or tissues denotes that such cells or tissues do not or no longer form part of a plant, an animal or human body.

The term “transfection” refers to the process of introducing a nucleic acid into an animal cell.

The term “photoresponsive”, “photosensitive”, “light sensitising” may be used interchangeably and refer to the capacity to respond to electromagnetic radiation, such as e.g. visible light.

The term “delivery yield” as used herein refers to the ratio of the quantity of living (viable) cells comprising one or more (macro) molecules after performing the method as taught herein (e.g. the quantity of living cells comprising the one or more (macro) molecules as detected after the delivery method) relative to the quantity of living (viable) cells before performing the method as taught herein (e.g. the quantity of living cells as detected before the delivery method).

The viability of cells after performing the method as taught herein (%) may be determined by dividing the quantity, such as number, of viable cells obtained after performing the method as taught herein by the quantity, such as number, of (total) viable cells before performing the method as taught herein, followed by multiplying the resulting value by 100.

The efficiency of the method as taught herein (%) may be determined by dividing the quantity, such as number, of viable cells comprising the one or more (macro) molecules obtained after performing the method as taught herein by the quantity, such as number, of (total) viable cells obtained after performing the method as taught herein, followed by multiplying the resulting value by 100.

The term “particle” as used herein refers to a particle or a group, agglomerate, or cluster of two or more particles having dimensions (more particularly the largest dimensions of the particles) of about 1 nm to about 2000 nm (2 μm).

The term “microparticle” as used herein refers to a particle or a group, agglomerate, or cluster of two or more particles having dimensions (more particularly the largest dimensions of the particles) of more than 1000 nm (>1 μm) and at most 2000 nm (≤2 μm).

The term “nanoparticle” refers to a particle or a group, agglomerate, or cluster of two or more particles having dimensions (largest dimensions of the particles) of at least 1 nm (≥1 nm) and at most 1000 nm (≤1 μm).

The dimensions of a particle, for example a width, height or diameter of a particle, can be determined using Transmission Electron Microscopy (TEM), Scanning Electron Microscopy (SEM) or atomic force microscopy (AFM).

The term “chimeric antigen receptor” or “CAR” (also known as chimeric immunoreceptors, chimeric T cell receptors or artificial T cell receptors) refers to a receptor protein that has been engineered to give T cells the new ability to target a specific protein. The receptors are chimeric because they combine both antigen-binding and T-cell activating functions into a single receptor.

The terms “suspension” and “cell suspension” generally refer to a heterogenous mixture comprising cells dispersed in a liquid phase. As the mixture is generally liquid, the cells may in principle be able to, but need not, settle or sediment from the mixture.

Cells such as animal cells including human cells may be “adherent”, i.e., require a surface for growth, and typically grow as an adherent monolayer on said surface (i.e., adherent cell culture), rather than as free-floating cells in a culture medium (suspension culture). Adhesion of cells to a surface, such as the surface of a tissue culture plastic vessel, can be readily examined by visual inspection under inverted microscope. Cells grown in adherent culture require periodic passaging, wherein the cells may be removed from the surface enzymatically (e.g., using trypsin), suspended in growth medium, and re-plated into new culture vessel(s). In general, a surface or substrate which allows adherence of cells thereto may be any substantially hydrophilic substrate. As known in the art, tissue culture vessels, e.g., culture flasks, well plates, dishes, or the like, may be usually made of a large variety of polymeric materials, suitably surface treated or coated after moulding in order to provide for hydrophilic substrate surfaces.

The phrase “generation of a vapour bubble” includes expansion of the vapour bubble, collapse of the vapour bubble, or a combination of expansion and collapse of the vapour bubble, and secondary effects that can be the result of the bubble expansion and collapse, such as pressure waves and flow of the surrounding medium. The terms “vapour bubble” or “bubble” as used herein refer to vapour nanobubbles and vapour microbubbles. Preferably, a vapour bubble may have a diameter in the range of 10 nm to 100 μm. Vapour bubbles may comprise water vapour bubbles.

The terms “subject”, “individual” or “patient” can be used interchangeably herein, and typically and preferably denote humans, but may also encompass reference to non-human animals, preferably warm-blooded animals, even more preferably mammals, such as, e.g., non-human primates, rodents, canines, felines, equines, ovines, porcines, and the like. The term “non-human animals” includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In certain embodiments, the subject is a non-human mammal. In certain embodiments, the subject is a human subject. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species.

Suitable subjects may include without limitation subjects presenting to a physician for a screening for a disease or condition, subjects presenting to a physician with symptoms and signs indicative of a disease or condition, subjects diagnosed with a disease condition, and subjects who have received an alternative (unsuccessful) treatment for a disease or condition.

The term “therapeutically effective amount” refers to an amount of an active compound, such as the T cells as taught herein, that when administered brings about a positive therapeutic response with respect to treatment of a patient having the disease or condition being treated.

The terms “pharmaceutical composition”, “pharmaceutical formulation” or “pharmaceutical preparation” may be used interchangeably herein and refer to a mixture comprising an active ingredient. The terms “composition” or “formulation” may likewise be used interchangeably herein.

Pharmaceutical compositions as intended herein may be formulated for essentially any route of administration, such as without limitation, oral administration (such as, e.g., oral ingestion), parenteral administration (such as, e.g., subcutaneous, intravenous or intramuscular injection or infusion), and the like.

The term “homeostasis” as intended herein refers to the state of steady internal, physical, and chemical conditions maintained by a cell. This is the condition of optimal functioning for the cell and includes many variables being kept within certain pre-set limits. Variables include but are not limited to pH of extracellular fluid, concentrations of sodium, potassium and calcium ions. In a specific embodiment as described herein, the term ‘homeostasis’ refers to an unaltered state of the following markers, provided that said levels are not influenced by the cargo that is brought into the cell: levels of inflammatory cytokines in a time frame of 0 h to 24 h after photoporation, said cytokines are chosen from tumour necrosis factor (TNF), interferon γ (IFNγ), IL-5, IL-6, IL-9, IL-10, IL-13 and IL-17A. Other markers include CD137, CD154 and PD1, again provided that said levels are not influenced by the cargo that is brought into the cell.

In a first aspect, the invention relates to a T cell, wherein the homeostasis of said T cell within at least 24 h after photoporation is unaffected and comparable to the homeostasis prior to said photoporation or compared to a non-photoporated T-cell. In a further embodiment, said homeostasis is unaffected for a time period of at least 1 h, 2 h, 3 h, 4 h, 5 h; 6 h, 7 h, 8 h, 9 h, 10 h, 11 h, 12 h, 13 h, 14 h, 15 h, 16 h, 17 h, 18, 19 h, 20 h, 21 h, 22 h, 23 h, 24 h, up and including to 48 h.

Photoporation was identified as a suited technique for engineering T-cells while causing a minimal of impact to the nature of the cells.

Note that photoporation results in the formation of pores in the cell membrane through which metabolites and ions can migrate. Said migration depends on the presence of a concentration gradient between the intracellular environment and the extracellular environment. Components that are present in the cell at a higher concentration than the environment (eg the cell medium) will migrate towards the extracellular environment. Alternatively, compounds that reside at higher levels in the extracellular environment will cause an influx in the cell. Consequently, said photoporated cell differs from the cells normally found in nature.

In an embodiment, said photoporation occurred by means of photoresponsive organic or inorganic nanoparticles. The term ‘nanoparticle’ refers to particles having an equivalent spherical diameter ranging between 1 nm and 1000 nm. Said particles may have any shape. They may for example be spherical, elliptical, rod-like shaped, pyramidal, branched, or may have an irregular shape. Said particles may comprise individual particles or a combination or cluster of two or more particles positioned close to each other.

The dimensions of a particle, for example a width, height or diameter of a particle, can be determined using Transmission Electron Microscopy (TEM), Scanning Electron Microscopy (SEM) or atomic force microscopy (AFM).

The size of the particles is preferably defined by the equivalent spherical diameter d (also referred to as the equivalent volume diameter).

In an embodiment, said particles are embedded in a structure. The material of the structure into which the particles able to absorb electromagnetic radiation are embedded comprises for example an inorganic material or an inorganic based material, for example silica or a silica based material or a ceramic or ceramic based material. In another embodiment, said material is an organic material or organic based material, such as a carbon or carbon based material or a polymer or polymer based material. The material of the structure may also comprise a composite material comprising at least one of the above mentioned materials, for example, a composite material comprising an organic and an inorganic material. Preferred materials of the structure comprise or are based on polystyrene, polycaprolacton, ethylcellulose, cellulose acetophthalate, polylactic acid, polylactic-co-glycolic acid, cellulose, polyvinylalcohol, polyethylene glycol, gelatin, collagen, silk, alginate, hyaluronic acid, dextran, starch, polycarbonate or polyacrylate.

In an embodiment the structure comprises a surface modified material, for example a surface modified polymer material. The surface modification comprises for example the application of a coating (for example collagen) to enhance cell attachment to the material of the structure.

In an embodiment said photoresponsive nanoparticles are embedded in a solid structure, such as fibers or a combination of fibers.

In an embodiment said solid structure comprises a non-porous structure such as a polymer sheet or polymer foil. A particular preferred embodiment comprises a polymer sheet comprising or based on polystyrene, polycaprolacton, ethylcellulose, cellulose acetophthalate, polylactic, polylactic-co-glycolic acid, cellulose, polyvinylalcohol, polyethylene glycol, gelatin, collagen, silk, alginate, hyaluronic acid, dextran, starch, polycarbonate or polyacrylate. The polymer sheet comprises for example iron oxide particles and/or carbon particles embedded in the polymer sheet.

In another embodiment said solid structure comprises a porous polymer structure such as structures comprising fibres (for example polymer fibres), structures comprising particulates (for example polymer particulates), structures comprising a combination of fibres and particulates (for example a combination of polymer fibres and/or polymer particulates) and structures comprising foam (for example polymer foam).

In a particularly preferred embodiment said solid structure comprises photothermal electrospun nanofibers.

The term “electrospun nanofibers” as used herein refers to nanofibers produced according to an electrospinning production method, wherein electrospinning is a fiber production method that uses electric force to draw charged threads of polymer solutions or polymer melts up to fiber diameters in the order of some hundred nanometers.

The structure may comprise a porous or non-porous structure. Porous structures can be preferred as they have the advantage to have a high free area surface and thus have a large surface available to be exposed to cells that are introduced on or near the structure according to the method of the present invention. Preferably, the porous structure has a pore size that allows partial or complete penetration of the cells introduced on or near the structure into the pores. Preferably, the porous structure has a pore size that does not restrict access of molecules present in the cell medium to the cells. The porosity of a structure is defined as the ratio of the volume of the pores or voids of a structure over the total volume occupied by that structure, i.e. the sum of the volume V of the structure (the volume of the material and the particles embedded in the material) and the volume of the pores or voids of that structure. The porosity may range between 0% and 100%. In case the structure comprises a porous structure the porosity of the structure is preferably at least 50%, at least 60% at least 80%, at least 90%, at least 95% or at least 99%.

Said photoresponsive inorganic particle may comprise metal particles, metal oxide particles, carbon or carbon based particles, particles comprising one or more light absorbing compounds or particles loaded or functionalized with one or more light absorbing compounds.

Examples of metal particles comprise gold particles, silver particles, platinum particles, palladium particles, copper particles and alloys thereof. Preferred metal particles comprise gold particles, silver particles and alloys thereof.

Examples of metal oxide particles comprise iron oxide, titanium oxide, zirconium oxide, cerium oxide, zinc oxide and magnesium oxide.

Examples of carbon or carbon based particles comprise graphene quantum dots, (reduced) graphene oxide and carbon nanotubes.

Examples of particles comprising one or more light absorbing compounds or particles loaded or functionalized with one or more light absorbing compounds comprise particles comprising, loaded or functionalized with synthetic organic or inorganic absorbers as well as particles comprising, loaded or functionalized with naturally occurring absorbers or derivatives thereof. Particular examples comprise liposomes, solid lipid nanoparticles, polymer based particles comprising loaded or functionalized with light absorbing dye molecules such as indocyanine green, inorganic quantum dots (having low fluorescence quantum yield), naturally occurring light absorbers like pigments (such as melanin, rhodopsin, photopsins or iodopsin) and synthetic analogs like polydopamine, or photosensitizers used in photodynamic therapy.

Said photoresponsive organic particle may be a photoresponsive polymer-based particle. In a further embodiment, the photoresponsive organic particle may be a photoresponsive polymer-based particle selected from a polydopamine (PD) particle, a poly(N-phenylglycine) (PNPG) particle, a poly-2-phenyl-benzobisthiazole (PPBBT) particle, a porphyrin particle, a phthalocyanine particle, or a polypyrrole particle. In further embodiments, the photoresponsive organic particle may comprise or consist of polydopamine, poly(N-phenylglycine), poly-2-phenyl-benzobisthiazole, porphyrin, phthalocyanine or polypyrrole. In further preferred embodiments, the photoresponsive organic particle may be prepared (produced or synthetised) from a clinically approved monomer, such as dopamine hydrochloride, thereby facilitating clinical transition of the methods as taught herein for the production of engineered therapeutic cell products, e.g. CAR-T cells.

In another embodiment the photoresponsive organic particle may be a polymer-based particle, a protein-based particle, a lipid-based particle (e.g. liposome or solid lipid particle), or a combination thereof comprising a light absorbing molecule. In further embodiments, the photoresponsive organic particle may be a polymer-based particle comprising a light absorbing molecule. In embodiments, the photoresponsive organic particle may be a protein-based particle comprising a light absorbing molecule. In further embodiments, the photoresponsive organic particle may be a lipid-based particle comprising a light absorbing molecule. In further preferred embodiments, the photoresponsive organic particle may be a solid lipid particle comprising a light absorbing molecule. In embodiments, the photoresponsive organic particle may be a combination of two or more of a polymer-based particle, a protein-based particle, a lipid-based particle comprising a light absorbing molecule. Such photoresponsive organic particles may be prepared using clinically approved molecules, thereby facilitating clinical transition of the delivery methods as taught herein for the production of engineered therapeutic cell products, such as CAR-T cells. In further embodiments of the photoresponsive organic particle may be a polymer-based particle, a protein-based particle, a lipid particle loaded with or functionalized with a light absorbing molecule. In embodiments the photoresponsive organic particle may be a polydopamine particle, preferably a polydopamine particle coated with albumin.

In an embodiment said photoresponsive organic particles may be present as individual particles, for instance in an aqueous solution, such as in a cell culture medium. In another embodiment the photoresponsive organic particles may comprise a group, agglomerate, or cluster of two or more particles, for instance in an aqueous solution, such as in a cell culture medium. Said particles, group of particles, agglomerate or cluster may have any shape. For example, said particles, group of particles, agglomerate or cluster may be spherical, elliptical, rod-like shaped, pyramidal, branched, or may have an irregular shape.

The term “based” as used in the context of the material of the organic particle as defined above is to be understood as a particle that predominantly comprises or is made of said material. In other words, said protein-based particle is to be understood as a particle that mainly comprises or completely consists of one or more proteins or peptides. A lipid-based or “lipid particle” may be used interchangeably herein and refer to particles comprising, consisting essentially of, or consisting of one or more lipids.

In a preferred embodiment, said photoporation is photothermal electrospun nanofibres (PEN) photoporation.

In an embodiment, said photoporation introduces one or more (macro) molecules. In a further embodiment, said (macro) molecule are otherwise not present in a native T cell prior to photoporation. In another embodiment, the amount of one or more (macro) molecules present in said T cell after photoporation is higher than prior to photoporation.

Said one or more (macro) molecules is preferably selected from the group of a nucleic acid, a protein, a peptide, a chemical substance, a polysaccharide, or any combination thereof. In a further preferred embodiment a combination of said one or more (macro) molecules may be a gene editing system e.g. CRISPR/Cas system. In an embodiment, said macromolecule may be a nucleic acid such as DNA or (m) RNA encoding a CAR. In an embodiment, the one or more (macro) molecules may be a negatively charged protein at physiological pH (e.g. pH of about 6 to about 8). (IEP). In further preferred embodiments, the one or more (macro) molecules may be a neutral protein at physiological pH (e.g. pH of about 6 to about 8).

In an embodiment, the molecular weight of said one or more macromolecules is at least 100 Da, such as between 0.1 and 5000 kDa. In an embodiment the molecular weight of said one or more (macro) molecules is at most 1000 kDa, more preferably at most 500 kDa. In embodiments, said one or more (macro) molecules may be a nucleic acid, such as (m) RNA or (plasmid) DNA, having a size of at least 0.5 kilobase (kb). For example, the one or more (macro) molecules may be a nucleic acid, such as (m) RNA or (plasmid) DNA, having a size of at least 0.6 kb, at least 0.7 kb, at least 0.8 kb, at least 0.9 kb, at least 1.0 kb, at least 1.5 kb, at least 2.0 kb, or more. For example, the one or more (macro) molecules may be a nucleic acid, such as (m) RNA or (plasmid) DNA, having a size of at least 3.0 kb, at least 4.0 kb, at least 5.0 kb, at least 6.0 kb, at least 7.0 kb, at least 8.0 kb, at least 9.0 kb, at least 10.0 kb, or more.

In an embodiment, the one or more (macro) molecules may be a protein, a polysaccharide, or combination thereof.

Cells to be used for photoporation, as described in detail above, may be suitably cultured or cultivated in vitro. Said cells may be isolated cells or tissues. The terms “culturing” or “cell culture” are common in the art and broadly refer to maintenance of cells and potentially expansion (proliferation, propagation) of cells in vitro. Typically, animal cells, such as mammalian cells, such as human cells, are cultured by exposing them to (i.e., contacting them with) a suitable cell culture medium in a vessel or container adequate for the purpose (e.g., a 96-, 24-, or 6-well plate, a T-25, T-75, T-150 or T-225 flask, or a cell factory), at art-known conditions conducive to in vitro cell culture.

As mentioned, the homeostasis of the T-cell after photoporation remained largely unaltered. This was further reflected by minimal or no changes in cell size, calcium levels, proliferation and marker profiles of the cells. In an embodiment the cell size of said T cell within at least 24 h or within 24 h after photoporation differs maximally 3%, preferably maximally 2% and most preferably maximally 1% compared to the cell size of said T cell prior to photoporation or compared to a non-photoporated T cell. Said cell size can be measured by conventional means in the art, such as by means of microscopy. In an embodiment, confocal microscopy is used, wherein cells are labelled (eg by calcein AM) and cell size is subsequently was measured by confocal imaging with a 10× objective. Image processing can be used as the area of the cells in the image.

In an embodiment the calcium level in said T-cell in an interval of 0 to 24 h, or within at least 24 h after photoporation, differs maximally 2%, preferably maximally 1.5%, more preferably maximally 1.25% and most preferably maximally 1% compared to the calcium level of said T cell prior to photoporation or compared to a non-photoporated T cell. Intracellular Calcium levels were measured using a Fluo-4 Direct™ Calcium Assay Kit (#F10471, Invitrogen) according to the manufacturer's instructions.

In an embodiment, no significant increase in inflammatory cytokines is detectable for at least 24 h, or in a time frame of 0 h to 48 h, more preferably 24 h to 48 h after photoporation. In an embodiment, said inflammatory cytokines are chosen from the group of TNF, IFNγ, IL-5, IL-6, IL-9, IL-10, IL-13 or IL-17A.

In an embodiment, the proliferation N/N0 of the photoporated T cell in a timeframe of 0 to 72 h is similar to that of a non-photoporated T cell. In another embodiment the proliferation N/N0 of the photoporated T cell in a time interval up to 72 hours after photoporation increases from at least 1 to at least 2, preferably from at least 1 to at least 3, more preferably from at least 1 to at least 4 and most preferably from at least 1 to at least 5. Cell proliferation comprises an increase in the number of cells as a results of cell growth and cell division. Consequently, a cell proliferation N/N0 is a measurement of high cell viability and unaltered cell homeostasis. A healthy cell proliferation N/N0 comprises an exponential growth similar to cell proliferation of non-photoporated T cells. Subsequently, a photoporated T cell according to the embodiment described above is able to proliferate exponentially indicates unaltered cell homeostasis and high cell viability after photoporation.

The marker profile of the photoporated cell remains largely unchanged. In an embodiment the photoporation did not result in an upregulation of CD137, PD1 and/or CD154 within at least 24 h or within 48 h after photoporation compared to the levels prior to said photoporation. The markers CD137, PD1 and CD154 are activation markers. Upregulation of said markers after photoporation points towards unwanted phenotypical changes of the T cell. Subsequently, an unchanged marker profile is again confirmation an unaltered cell homeostasis after photoporation.

In an embodiment said T cell is a CAR T cell. In a further embodiment of the invention said CAR T cell after photoporation has maintained a similar tumour cytolytic capacity as its non-photoporated counterpart. In a further embodiment said tumour cytolytic capacity is similar for an effector-to-target ratio of at least 5/1, preferably at least 4/1, more preferably at least 3/1, even more preferably at least 2/1 and most preferably at least 1/1.

The term “similar tumour cytolytic capacity” as used herein refers to a similarity of at least 75%, preferably at least 90%, more preferably at least 95% and most preferably at least 99% of a tumour cytolytic capacity defined for an effector/target ratio of at least 5/1, wherein the effector is a T cell and the target is a tumour cell.

In an embodiment said CAR T cell is engineered such that it targets at least one of the following molecules chosen from CD70, TNFRSF17, ILR3A, SDC1, EGFRvIII, MUC1, FAP, CD44, CD19, MS4A1, CD22, EPCAM, PDCD1, CA9, CD174, TNFRSF8, CD33, CD38, EPHA2, CD274, FOLR1, SLAMF7, CD5, NCAM1, ERBB2, KDR, L1CAM, GD2, ULBP1, ULBP2, IL1RAP, GPC3, IL13RA2, ROR1, CEACAM5, MET, EGFR, MSLN, FOLH1, CD23, CD276, CSPG4, CD133, TEM1, GPNMB, PSCA.

Also disclosed herein is a population of T cells or a pharmaceutical composition comprising the T cells as described herein. Preferably, a composition or population comprising said T cells as disclosed herein may comprise at least 10³, 10⁶, 10⁹ or more cells (for example, between 5 million and 500 million or between 5 million and 250 million or between 50 million and 500 million or between 50 million and 250 million or between 100 million and 500 million or between 100 million and 250 million of cells for each dose or administration).

Such compositions or populations may also include other agents of biological (e.g. antibodies or growth factor) or chemical origin (e.g. drugs, cell preserving or labelling compounds) that may provide a further therapeutic, diagnostic, or any other useful effect. The literature provides several examples of optional additives, excipients, vehicles, and/or carriers that are compatible with cell-based pharmaceutical compositions that may include further specific buffers, growth factors, or adjuvants, wherein the amount of each component of the composition is defined (in terms of micrograms/milligrams, volume, or percentage), as well as the means to combine them with liver progenitor cells.

In an embodiment, said pharmaceutical composition may comprise one or more pharmaceutically acceptable carriers, excipients s and/or diluents. The pharmaceutically acceptable carrier, excipient and/or diluent is thus chosen such that the cells as described herein remain viable and retain their properties. The carrier can be a pharmaceutically acceptable solvent or dispersing medium containing, for example, water, saline, phosphate buffered saline, polyol (for example, glycerol, propylene glycol, liquid polyethylene glycol, and the like) and suitable mixtures thereof.

In practicing the methods of treatment or uses provided herein, a therapeutically effective amount of pharmaceutical composition described herein is administered to a mammal having a disease, disorder, or condition to be treated. In some embodiments, the mammal is a human. In other embodiments, the mammal is non-human. A therapeutically effective amount may vary widely depending on the severity of the disease, the age and relative health of the subject, the potency of the therapeutic agent used and other factors. The therapeutic agents, and in some cases, compositions described herein, may be used singly or in combination with one or more therapeutic agents as components of mixtures.

An issue concerning the therapeutic use of the cells as described herein is the quantity of cells necessary to achieve an optimal effect. Doses for administration may be variable and may include an initial administration followed by subsequent administrations and can be ascertained by the skilled artisan armed with the present disclosure. Typically, the administered dose or doses will provide for a therapeutically effective amount of the cells, i.e., one achieving the desired local or systemic effect and performance. In addition, the skilled person can readily determine the optional additives, vehicles, and/or carrier in pharmaceutical compositions of the invention to be administered to a subject.

In some embodiments, the pharmaceutical composition described herein includes at least one additional active agent described herein. In some embodiments, the at least one additional active agent is a chemotherapeutic agent, cytotoxic agent, cytokine, growth-inhibitory agent, anti-hormonal agent, anti-angiogenic agent, or checkpoint inhibitor. In some embodiments, the at least one additional active agent is an adjuvant for increasing effectiveness of vaccination.

The pharmaceutical composition or population must be sterile and stable under the manufacturing and storage conditions. The composition can be formulated as a solution, microemulsion, dispersion, in liposomes or in other ordered structures that are suitable for this purpose and know by the artesian.

The pharmaceutical composition or population described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, immediate release formulations, controlled release formulations, delayed release formulations, extended-release formulations, pulsatile release formulations, multi-particulate formulations, and mixed immediate.

In a further embodiment the compositions or populations as described herein can be used in therapeutic methods for in vivo administration (in humans or in animal models) or in vitro applications either as fresh or in formulation suitable for long-term storage (e.g. cryopreserved cells). These pharmaceutical compositions can be provided in a format that is appropriate for the desired method of treatment, the selected route of administration, and/or storage, as well as in the preferred means for providing such pharmaceutical compositions (e.g. within a kit). Other agents of biological (e.g. antibodies or growth factor) or chemical origin (e.g. drugs, preserving or labelling compounds) that may provide any other useful effect can be also combined in such compositions.

In a final aspect methods for treatment and therapeutic use are described herein, based on the T cells, populations and compositions as described above.

Cells such as T cells may be obtained from (e.g., isolated from, derived from) a biological sample, preferably a biological sample of a mammalian subject.

The term “biological sample” or “sample” as used herein refers to a sample obtained from a biological source, e.g., from an organism, an animal or human subject, cell culture, tissue sample, etc. A biological sample of an animal or human subject refers to a sample removed from an animal or human subject and comprising cells thereof. The biological sample of an animal or human subject may comprise one or more tissue types and may comprise cells of one or more tissue types. Methods of obtaining biological samples of an animal or human subject are well known in the art, e.g., tissue biopsy or drawing blood.

In an embodiment the T cells, population or composition can be used to treat a wide range of diseases and conditions. Essentially any disease that involves the specific or enhanced expression of a particular antigen can be treated by targeting T cells to the antigen. Examples include autoimmune diseases, infections, and cancers can be treated with T cells, populations and/or compositions of the invention. These include cancers, such as primary, metastatic, recurrent, sensitive-to-therapy, refractory-to-therapy cancers (e.g., chemo-refractory cancer). The cancer may be of the blood, lung, brain, colon, prostate, breast, liver, kidney, stomach, cervix, ovary, testes, pituitary gland, esophagus, spleen, skin, bone, and so forth (e.g., B-cell lymphomas or a melanomas). In the case of cancer treatment T cells typically target a cancer cell antigen, also known as a tumor-associated antigen.

In an embodiment the T cells, population or composition is used to treat a subject having minimal residual disease such as cancer patients that are in apparent remission. Using new highly sensitive diagnostic techniques, cancer-associated antigens (or cancer cells) can be detected in patients that do not exhibit overt cancer symptoms. Such patients may be treated by the instant methods to eliminate residual disease by use of antigen-targeted T cells. In preferred embodiments, CAR T cells are used. In a further embodiment the treatment further comprises expression of a membrane-bound proliferative cytokine, as these cells will retain the ability to expand in vivo despite the low amount to target antigen.

In an embodiment the T cells, population or composition can be used to treat cell proliferative diseases, fungal, viral, bacterial or parasitic infections. Pathogens that may be targeted include, with limitation, Plasmodium, trypanosome, Aspergillus, Candida, HSV, RSV, EBV, CMV, JC virus, BK virus, or Ebola pathogens. Further examples of antigens that can be targeted by T cells of the embodiments include, without limitation, CD19, CD20, carcinoembryonic antigen, alphafetoprotein, CA-125, 5T4, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD123, CD23, CD30, CD56, c-Met, meothelin, GD3, HERV-K, IL-11Ralpha, kappa chain, lambda chain, CSPG4, ERBB2, EGFRvIII, or VEGFR2.

In an embodiment the therapeutic use of T cells as described herein comprises stimulating a universal chimeric antigen receptor mediated immune response in mammals. Preferably the invention provides the use of T cells as a therapeutic treatment, more preferably as a treatment of cancer or an autoimmune disease. An autoimmune disease arises from an abnormal immune response of the body against substances and tissues normally present in the body (autoimmunity).

In an embodiment the therapeutic use comprises T cells expanded in vitro to provide a sufficient T cell-derived effector cell population that is attenuated for further proliferation in vivo in the subject receiving adoptive T cell therapy.

In a preferred embodiment of the invention the T cell, population or T-cell(s) of the composition is allogenic to the patient.

In a preferred embodiment of the invention the T cell, population or T-cell(s) of the composition is autologous to the patient.

In a preferred embodiment of the invention said patient has a cell proliferative disease.

In a further preferred embodiment of the invention said cell proliferative disease is autoimmune disease and wherein the T cell is targeted to autoimmune cells.

In a another preferred embodiment of the invention said cell proliferative disease is a cancer and wherein the T cell is targeted to a cancer-cell antigen.

In a preferred embodiment of the invention said patient is a human.

The invention is further described by the following non-limiting examples which further illustrate the invention, and are not intended to, nor should they be interpreted to, limit the scope of the invention.

### EXAMPLES

With as a goal illustrating better the properties of the invention the following presents an example and limiting in no way other potential applications of the current invention.

### Example 1: Using Photothermal Electrospun Nanofibers (PENs) for Safe and Efficient Intracellular Delivery of (Macro) Molecules into Cells

### Methods

### Fabrication of Photothermal Nanofibers.

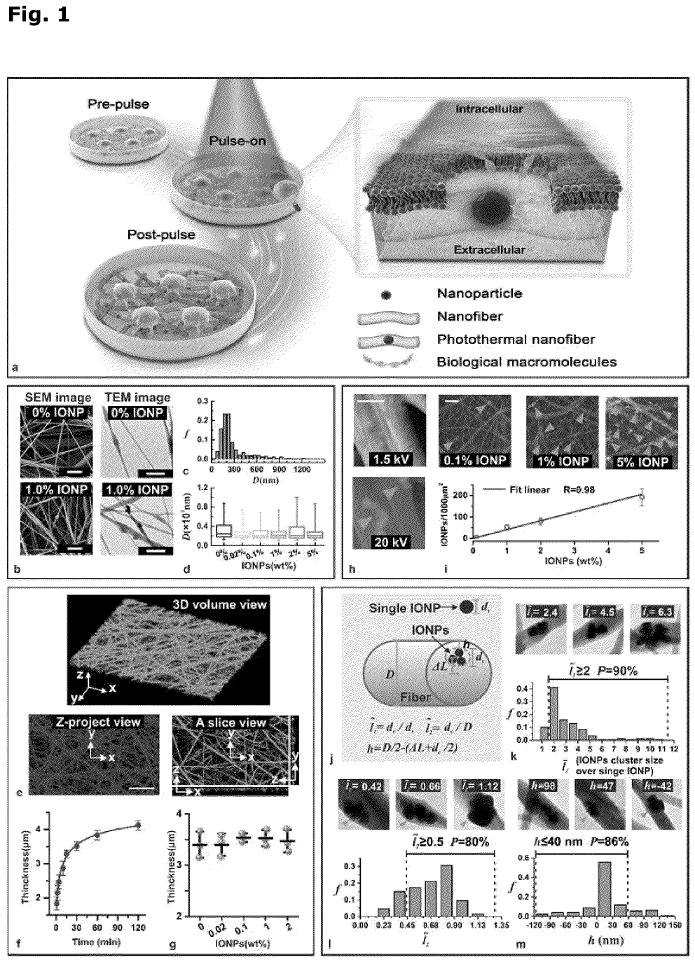

Polycaprolactone (PCL, Molecular Weight=˜70,000 g/mol), N, N-Dimethylformamide (DMF), Tetrahydrofuran (THF) and iron oxide (Fe3O4) nanopowder (#MKBW3262, Sigma-Aldrich, Belgium) were purchased from Sigma-Aldrich (Belgium). The iron oxide nanopowder was re-dispersed in 2 ml of a 1:1 DMF/THF solution to which 480 mg of dried PCL was added. This mixture was used for electrospinning of fibers which were collected on microscope glass slides (#1000912, Marienfeld, Germany) mounted on a grounded rotating collector (FIG. 1). During electrospinning, unless otherwise specified, the applied voltage, flow rate and electrospinning distance were fixed at 10 kV, 0.3 mL/h and 20 cm, respectively. The grounded rotating collector was set at a rotating speed of 500 rpm. After 30 min (or specifically indicated time) the electrospinning process was stopped and glass slides with the nanofiber web were separated from the rotating collector and sterilized by UV irradiation for 45 min in a laminar flow cabinet.

### Fabrication of Home-Made PEN Cell Culture Substrates.

8-well Secure-Seal™ double sided adhesive spacers (#S24737, Invitrogen) were sterilized by UV irradiation for 45 min in a laminar flow cabinet. After removing the protective sealing from one side of the adhesive spacers, they were gently stuck on a nanofiber web. Next, these samples were immersed in DI water for 3 min for easy removal of the web (with adhesive spacers on top) from the glass slides. The web was manually cut into smaller pieces with either one or 4 adhesive wells per piece (into which cells can be grown) and stored in PBS buffer.

Next, these PEN cell culture substrates were further modified with collagen for optimal cell. Poly(allylamine hydrochloride) (PAH, Mw=17,560 g/mol, #MKBZ2824V, Sigma-Aldrich, Bornem, Belgium) and concentrated sulfuric acid solution (96%) were purchased from Sigma-Aldrich. Collagen I Rat Protein was purchased from Thermo Fisher Scientific (#A1048301, Gibco™, Belgium). 4-well PEN cell culture substrates were immersed in 32% sulfuric acid solution (3 mL per well of 6-well plate) for 3 min. After washing with distilled water, they were immersed into an aqueous solution of the polyelectrolyte PAH (2 mg/mL, 0.5 M NaCl) for 15 min and rinsed 3 times with distilled water. Physisorption of PAH to the nanofiber surface made the fibers positively charged. Next, the PAH coated fibers were immersed in a 0.5 mg/mL aqueous solution of Collagen I Rat Tail Protein for 15 min and rinsed with PBS solution. Hydrated fibers were formed through surface hydrolysis, for which PCL-fibers were soaked for more than 1 hour in 0.1 M NaOH at 4° C. and rinsed with PBS solution. Finally, the modified PEN substrates were stored in PBS before further use.

### Culturing or Collecting Hela and Jurkat Cells in the PEN Cell Substrates for Photoporation Treatment.

Hela cells (#CCL-2) and Jurkat clone E6.1 (#TIB-152) were obtained from ATCC and employed as model for the transfection of respectively adherent and suspension cells by PEN photoporation. Human lung epithelial cells (H1299) stably expressing enhanced green fluorescent protein (GFP) were used for the validation of siRNA knockdown experiments. HeLa cell culture medium was made from DMEM/F-12 with 2 mM glutamine, 100 U/mL penicillin/streptomycine and 10% heat-inactivated fetal bovine serum (FBS). H1299 and Jurkat cell culture medium consisted of RPMI1640 with 2 mM glutamine, 100 U/mL penicillin/streptomycine and 10% FBS.

To grow adherent cells, PEN cell culture substrates were placed in 6-well titer plates (#10062-892, VWR) to which HeLa or H1299 were added (˜1×10⁶ cells in 2 mL cell culture medium). Cells were allowed to attach and grow during 24 h in a cell incubator at 37° C. in a humidified atmosphere with 5% CO₂. Just prior to photoporation treatment, the molecules of interest that need to be delivered into the cells were added to the cell medium.

Jurkat cells were cultured in 75 cm² or 182.5 cm² flasks (#734-2313, #734-2315, VWR) at a cell density between 1×10⁵ and 1×10⁶ cells/mL. For photoporation, the molecules of interest were added to the cell medium and cells were transferred to the PEN cell substrates at ˜2×10⁵ cells/well. Cells were allowed to sediment on the fiber web during 5 min before starting the photoporation laser scanning.

### Laser Irradiation of Cells on PEN Substrates.

Photoporation requires cells to be irradiated with laser light. Here we used a custom-built optical set-up as previously reported with some minor modifications (Xiong, R. H. et al., ACS Nano, 8, 6288-6296 (2014); Xiong, R. H. et al., Nano Lett., 16, 5975-5986 (2016)). Briefly, a pulsed laser with 7 ns pulse duration was tuned at wavelength of 647 nm (Opolette™ HE 355 LD, OPOTEK Inc, CA) and applied to irradiate the PEN substrates. The collimated pulsed laser beam was directed through a 1° Light Shaping Diffuser (Physical Optics Corporation, Torrance, CA), which in combination with an achromat lens in front of the microscope entrance and a 20× objective lens (Plan Fluor, Nikon) resulted in a laser beam diameter of ˜250 μm at the sample. The laser pulse energy was monitored by an energy meter (J-25 MB-HE&LE, Coherent) synchronized to the pulsed laser. In order to scan all the cells on the PEN substrates (diameter of ˜9 mm), a motorized microscope stage was used to scan the sample through the stationary laser beam line by line. As the laser repetition rate was 20 Hz, the scanning speed was set at 3 mm/s with a distance between subsequent line of 0.15 mm. In this way, all cells received at least one laser pulse up to maximally 4 in the overlapping regions between neighboring irradiation zones. In some experiments with Jurkat or human T cells, the cells were scanned multiple times, as indicated in the main text. In that case the cells were re-suspended within the PEN well and allowed to sediment again between each scan in order to let the cells randomly attach to the nanofibers at new locations.

### IONP-Sensitized Traditional Photoporation of Cells.

Polyethyleneimine (PEI) functionalized iron oxide nanoparticles (IONPs) were prepared by dispersing 100 mg of iron oxide powder (Iron Oxide Fe₃O₄ Nanopowder, #MKBW3262, Sigma-Aldrich, Belgium) in a 10 mL solution of 10 wt % branched PEI (bPEI, 25 kDa, Sigma-Aldrich) immediately followed by sonicated for 1 minute with a tip sonicator (10% A, Branson Digital Sonifier, Danbury, USA). The mixture was then further sonicated with a bath sonicator (Branson 2510 Branson Ultrasonics, Dansbury, CT, USA) for an additional 1 hour and then vigorously stirred overnight to allow PEI molecules to absorb on the surface of IONPs. Next, the unbound bPEI was removed by performing several washing steps with HyClone water (VWR) via centrifugation (4000×g, 10 minutes). Finally, PEI-coated IONPs with an appropriate size were selected via differential centrifugation. The physicochemical characterization (i.e., hydrodynamic diameter, zeta-potential and particle concentration) was performed respectively with dynamic light scattering (DLS, Zetasizer Nano-ZS, Malvern instruments Co., Ltd) (=hydrodynamic diameter and zeta-potential) and/or with Nanoparticle Tracking Analysis (NTA, NanoSight LM10, Malvern Panalytical, UK) (=hydrodynamic diameter and particle concentration).

For IONP-sensitized photoporation, Hela cells were grown in a 96-well plate (#10062-900, VWR®, US) at a density of 1×10⁴ cells per well. Next, cells were incubated for 30 min at 37° C. with PEI coated IONPs at various concentrations as indicated. Cells were subsequently photoporated at the indicated laser fluence in the presence of 2 mg/mL FD10 dissolved in cell culture medium.

### Detection of Vapour Nanobubbles.

The generation of vapour nanobubbles was detected by dark-field microscopy as they efficiently scatter light. As VNBs typically have a very short lifetime (<1 μs), depending on their size, we synchronized the camera (EMCCD camera, Cascade II: 512, Photometrics, Tucson, USA) with the pulsed laser by an electronic pulse generator (BNC575, Berkeley Nucleonics Corporation, CA, USA). The pulse laser sends a Q-switch signal to trigger pulse generator and it will trigger the camera at a setting delay.

### Detection of Reactive Oxygen Species (ROS).

ROS formation was evaluated with the probe 2′, 7′-Dichlorofluorescin (DCFH) as a fluorescence indicator. Briefly, DCFH was prepared by mixing 0.5 mL of 1 mM DCFH-DA (2′, 7′-Dichlorofluorescin diacetate, purchased from Sigma (#D6883) in methanol with 2.0 mL of 0.01 N NaOH for 30 min at room temperature. The mixture was neutralized with 10 ml of 25 mM NaH₂PO₄ to PH 7.2. All reactions were performed in 40 mM Tris-HCl in a total volume of 1 mL containing 25 μl DCFH solution and 10 μM Fe²⁺ (from FeSO₄).

To measure the amount of ROS generation by laser irradiation of the PEN substrates, 150 μl DI water was added to the PEN wells before starting the laser scanning procedure. After treatment, the DI water was collected again from the PEN wells and added to the DCFH solution. A negative control was included which did not receive laser treatment, while a positive control sample was prepared from 150 μl H₂O₂ added to the DCFH solution.

After further incubation for 2 h at 37° C., fluorescence was measured by a Victor3 microplate reader (#1420-040, PerkinElmer, Turku, Finland) with excitation at 485 nm and emission at 535 nm. Relative fluorescence intensity (FI) was calculated by equation (1):

$$Relative FI = \frac{{FI}_{s} - {FI}_{BG}}{{FI}_{CTRL} - {FI}_{BG}} \qquad ( 1 )$$

Where FIₛ is the fluorescence intensity of the actual sample, FI_{BG} is the fluorescence intensity of the background which is just water as blank sample, and FI_{CTRL} is the fluorescence intensity of the DCFH solution.

### Electron and Confocal Microscopy.

For TEM imaging, the nanofibers were directly electrospun on carbon-coated Cu grids (200-mesh). Following laser irradiation of the nanofibers, they were visualized by a JEM 1400 plus transmission electron microscope (JEOL, Tokyo, Japan) operated at 20-60 kV. For SEM imaging, samples were first coated with 5 nm platinum using a Quorum Q150T ES sputter coater. Scanning electron microscope images were taken with a Zeiss Crossbeam 540 Electron Microscope using a SE2 detector at 20 kV.

For visualization by confocal microscopy, fluorescent PCL nanofibers were fabricated by electrospinning a PCL solution mixed with the fluorophores 3-(2-benzothiazolyl)-7-(diethylamino) coumarin (coumarin-6, #12779, Sigma-Aldrich). A confocal laser scanning microscope (C1si, Nikon, Japan) with 60× water lens (Plan Apo VC, Nikon) was used to image the fluorescent PCL nanofibers. HeLa and H1299 cells grown on PEN substrates were imaged by the C1si confocal with a 10× lens (CFI Plan Apochromat, Nikon). For confocal imaging of Jurkat cells, their plasma membrane was stained with 10 μg/mL deep red fluorescent CellMask (#C10046, ThermoFisher Scientific). A series of z-stack confocal images were acquired in two channels (green channel recorded for nanofibers and deep red channel for the cells) with the 60× water lens.

### Quantification of Intracellular Delivery by Flow Cytometry.

Photoporation efficiency was quantified by flow cytometry. For HeLa's we used 10, 40, 70, 150, and 500 kDa FITC-dextran or 10 kDa Alexa Fluor® 647 labelled dextran as model compounds, which were added to the cells at a final concentration of 2 mg/ml or of 0.5 mg/mL, respectively. Before 24 h laser treatment, 1 million HeLa cells in 2 mL cell culture medium were added to the 6-well plate containing 4 PEN subtract well dishes. After photoporation on the PEN substrates, HeLa or H1299 cells were detached by 0.25% trypsin-EDTA (Invitrogen, Belgium) treatment and collected by 5 min 300×g centrifugation. To collect Jurkat or Human CD3+ T cells, the PEN substrates were simply washed one or two times with PBS.

Next, collected cells were re-suspended in flow buffer (PBS supplemented with 5% FBS) and measured by flow cytometry (CytoFLEX Cytometer, Beckman Coulter, Belgium) until at least 10000 events were detected per sample. The cells loaded with FITC-dextran or Alexa Fluor® 488 labelled siRNA were excited with a 488 nm laser and fluorescence was recorded in the 525/40 channel. On the other hand, when the cells were loaded with Alexa Fluor® 647 labelled dextran or labelled with PD1APC antibody (see below), a 638 nm laser was used to excite the cells and the fluorescence was detected in the 660/10 channel.

The following antibodies were used for flow cytometry analysis of human CD3+ T cells: CD3 BV421 (Pacific blue), CD4 BB700 (PERCP-Cy5.5), CD8 APC-Cy7 and PD1APC (Invitrogen, Belgium). Briefly, T cells were washed with PBS (PBS, Gibco-invitrogen) and re-suspended in FACS buffer, supplemented with 5% bovine serum albumin, BSA (Sigma-Aldrich, Bornem, Belgium). After a 30 min incubation at 4° C. with the indicated antibodies, the cells were washed and analyzed by flow cytometry. Pacific blue and PERCP-Cy5.5 were excited with a 405 nm and 488 nm laser with filter of 450/50 and 690/50, respectively. APC-Cy7 and APC was excited 638 nm laser with filter of 660/20 and 780/60, respectively. Control samples are used to define the threshold for positive cell loading, where the threshold value is defined as the 95% level of controlled cells.

### Evaluation of Cell Viability.

Two methods were employed to evaluate cell viability. To visualize dead cells with confocal microscopy, or to exclude them from flow cytometry analysis, Calcein AM (#C3100MP, Invitrogen™) was used as a viability stain. Viable cells will be positive for calcein fluorescence, while dead cells will not. Before analysis, cells were incubated for 30 min at room temperature with Calcein AM. For more accurate quantification of cell viability, the CellTiter-Glo® Luminescent cell viability assay (#G7571, Promega, Belgium) was used, which is based on the quantitation of ATP. After photoporation treatment, cell culture medium was removed and 100 μL CellTiter-Glo reagent solution was added to each sample together with 100 μL fresh cell medium. The samples were put on a shaker at 100 rpm for 10 min at room temperature. Finally, 100 μL solution was again removed from each sample and transferred to 96 titer well plates (#655075, Greiner Bio-one, Germany) for analysis by a microplate reader (GloMax®, Progmega, Belgium).

### Quantification of Cell Loading and Viability by Imaging Process.

After laser treatment, 3-5 confocal images were acquired with a confocal laser scanning microscope (C1si, Nikon, Japan) using a 10× lens (CFI Plan Apochromat, Nikon, Badhoevedorp, The Netherlands). Each image consists of green fluorescence (viability) and red fluorescence (loading efficiency) channels. A Matlab (The matworks, Natick, MA, USA) program was written for automated quantification of cell loading and cell viability. Untreated cells are used to define the threshold for positive cell loading, where the threshold value is defined as the 95% level of untreated cells. Similarly, cells are considered as alive when the green fluorescence intensity is higher than the 95% level of dead cells.

### Quantitative Fe Assay Via ICP-MS/MS.

The determination of Fe by means of inductively coupled plasma-mass spectrometry (ICP-MS) is hampered by the occurrence of spectral interference. Therefore, tandem ICP-mass spectrometry (ICP-MS/MS) was used instead and interference-free conditions were obtained by relying on chemical resolution using a reactive gas mixture of NH₃/He (1:9). Method optimization revealed that a mass-shift approach, whereby Fe was monitored under the form of the reaction product ion Fe(NH₃)₂⁺ provided the best conditions. This method was used to evaluate the potential release of IONPs from the fibers in the presence or absence of cells. In the absence of cells, DI water was added to the PEN substrates and was collected after laser treatment. Samples with cells were prepared as described earlier. After laser irradiation, the cells were collected by washing with PBS or by trypsinizing in the case of suspension and adherent cells, respectively. Finally, 100 μL aqua regia (3:1 HCl/HNO₃) was added to the samples to digest the cells and other potentially present organic matter. The sample solutions were diluted 100 times with 2% HNO₃ to a final volume of 10 mL in metal-free tubes, adding Y as internal standard at a final concentration of 1 μg/L (1,000 mg/L Y standard stock solution, Inorganic Ventures, Christiansburg, VA, USA) to correct for instrument instability and/or signal drift. External calibration standards (0, 0.5, 1, 2.5, 5 and 10 μg/L Fe+1 μg/L Y) were prepared by appropriate dilution of a 1000 mg/L Fe standard stock solution (Inorganic Ventures, Christiansburg, VA, USA) in 2% HNO₃, mimicking the matrix of the sample solutions. During all steps of the sample preparation, the solutions were mixed thoroughly using a vortex mixer. The tandem ICP-MS instrument (Agilent 8800 triple-quadrupole ICP-MS, Agilent Technologies, Japan) was tuned on a daily basis for high sensitivity across the mass range and low oxide ion formation to achieve optimal conditions for the interference-free determination of Fe. The determination of Fe was based on external calibration with internal standardization for which the ⁵⁶Fe(NH₃)₂⁺ signal intensity was normalized using the ⁸⁹Y(NH₃)₆⁺ signal intensity. A methodological detection limit of 80 μg/L was determined by multiplying the instrumental background-equivalent concentration (BEC) by the dilution factor (100×).

### Simulations of PEN Photothermal Response.

Numerical simulations were performed to get a deeper understanding of the photothermal response of PEN fibers to nanosecond pulsed laser irradiation. First, the laser-induced heating of IONPs was computed using the Generalized Multiparticle Mie Theory (GMM). It provides a rigorous description of the interaction of electromagnetic waves with (aggregates of) spherical particles, whose composition is determined by the real and imaginary part of their dielectric constant. In the GMM method, scattered fields from each individual sphere are solved in terms of the respective sphere-centered reference systems. In order to solve multisphere-scattering through the Mie-type multipole superposition approach, the incident plane wave is expanded in terms of vector spherical wave functions in each of the sphere-centered coordinate systems, obtaining the total electromagnetic field incident upon each sphere in the particle cluster, which consist of two parts: (1) the initial incident plane wave and (2) the scattered waves from all other spheres in the aggregate. In a next step, a single field representation for the total scattering field from the aggregate as a whole by expanding it in vector spherical wave functions is generated. Finally, with the total scattered field available, and based on the analytical expressions for the amplitude scattering matrix of an aggregate of spheres, it is possible to derive a rigorous formula for other fundamental scattering properties such as extinction, absorption, and scattering cross sections. In all calculations presented in this work the dielectric function tabulated by Querry for iron oxide (magnetite) was employed. The calculations were performed for 160 nm particles in water (n=1.33) or PCL (n=1.46). As the GMM code is restricted to applications in homogeneous media, for calculations at the polymer-water interface we have used the effective medium approximation. Here, we considered that particles were immersed in a dielectric environment with an effective refractive index of n_{eff}=1.40, considering that half of the IONPs are exposed to the aqueous medium. The calculations for linear arrangements of IONPs were performed with an inter-particle distance of 1 nm. Heat transfer from IONPs to the nanofiber PCL matrix and to the surrounding medium was simulated by a commercial CFD (Computation Fluid Dynamics) software package (ANSYS FLUENT) which allows to numerically solve the heat transfer equation. The simulation procedure was as follows. A 3D geometry model was built with a simulation domain of 6 μm×6 μm×36 μm including a cylindric domain (diameter=0.32 μm, length=30 μm) to represent a nanofiber and a spherical domain (diameter=0.16 μm) representing a single IONP. The simulation domain is discretized into a grid with a total of 2.85 million elements (the smallest mesh size was 30 nm). The boundary conditions were set as infinite boundary conditions. The initial temperature of IONPs were set according to the Mie theory calculations discussed above. The IONPs temperature were maintained for 7 ns this was the duration of the laser pulses used in this work. PCL polymer specific heat and thermal conductivity were set at 1250 J/kg-K and 0.175 W/m-K respectively. For the water surrounding the fiber a specific heat of 4182 J/kg-K and a thermal conductivity of 0.6 W/m-K were used.

### Calculation of the Temperature Increasing of Bulk Water.

The total absorption energy by IONPs embedded in fibers was simply calculated as:

$${Q}_{IONPs} = {C}_{IONPs} \times {m}_{IONPs} \times Δ {T}_{IONP} \qquad ( 3 )$$

Here, C is the heat capacity of IONPs, m is the mass of IONPs in a one PEN web dish and ΔT is the single IONP temperature increase after laser irradiation which is calculated by IONPs absorption cross section multiplying with laser fluence. Here, we assume that all IONPs heat energy finally transfer to the surrounding water causing the bulk temperature increasing as calculating as following:

$$Δ {T}_{water} = \frac{{Q}_{IONPs}}{{C}_{wαter} \times {m}_{wαter}} .$$

siRNA Transfections for Downregulation of GFP in H1299.

For siRNA transfections of H1299 cells, twenty-one nucleotide siRNA duplexes targeting the enhanced green fluorescent protein (siGFP) and negative control duplexes (siCTRL) were ordered from Eurogentec (Seraing, Belgium).

siGFP:
sense strand =
5′-CAAGCUGACCCUGAAGUUCtt-3′;
antisense strand =
5′-GAACUUCAGGGUCAGCUUGtt-3′.
siCTRL:
sense strand =
5′-UGCGCUACGAUCGACGAUGtt-3′;
antisense strand =
5′-CAUCGUCGAUCGUAGCGCAtt-3′.

To quantify intracellular delivery after PEN photoporation, siCTRL duplex was labeled with Alexa Fluor® 488 (Eurogentec). Before 24 h laser treatment, 1 million H1299 cells in 2 mL cell culture medium were added to the 6-well plate containing 4 PEN subtract well dishes. The amount of siRNA was added to the cells in final concentration of 1 μM except specifically indicating.

For calculating siRNA gene silencing efficiency, GFP knockdown efficiency was calculated according to equation (4):

$$Knockdown efficiency ( \% ) = ( 1 - \frac{{FI}_{GFP} +}{{FI}_{NTC \_ GFP +}} ) \times 1 \; 00 \% \qquad ( 4 )$$

Here, FI_{GFP}+ is the percentage positive cells in fluorescence intensity treated with anti-GFP SIRNA and FI_{NTC_GFP}+ is the percentage positive cells in fluorescence intensity in nontreated control samples. The data obtained from flow cytometery was post-processed with the FlowJo software package (Treestar Inc, Ashland, USA).

### Intracellular Delivery of RNPs Cas9 for Knockout of GFP in H1299 Cells.

crRNA:tracrRNA duplexes were prepared by mixing individual crRNAs in a 1:1 molar ratio with tracrRNA, followed by heating at 95° C. for 5 minutes and annealing at room temperature for 5-10 minutes. Next, Cas9 RNP complexes were obtained by mixing either crRNA:tracrRNA duplexes in a 2.5:1 molar ratio with Cas9 endonuclease and allowing the complexes to assemble for at least 10 minutes at room temperature prior delivery. H1299 cells were seeded on the PEN cell culture substrates as described above prior to PEN photoporation. On the day of photoporation, Cas9 RNPs were prepared as described above. RNP complexes were diluted in Opti-MEM at a final concentration as indicated in the main text, and added to the cells followed by photoporation by laser scanning. Post laser treatment, the cells were washed once with DPBS−, supplied with new culture medium and further incubated at 37° C., 5% CO2 prior to analysis of GFP knockout by confocal microscopy or flow cytometry. RNP gene knockout efficiency was calculated by equation (5):

$$Knockout efficiency ( \% ) = ( 1 - \frac{{FI}_{RNP \_ GFP}}{{FI}_{NTC}} ) \times 1 \; 00 \% \qquad ( 5 )$$

Here, FI_{RNP_GFP} is the mean fluorescence intensity of cells treated with RNPs for knockout of eGPF and FI_{NTC} is the mean fluorescence intensity of non-treated cells.

### PEN Photoporation and Electroporation of Human Embryonic Stem Cells.

The H9 human embryonic stem cell (hESC) line (WA09, WiCell, feeder free cultures were obtained via prof. C. Verfaillie, KULeuven, Belgium) was employed for all PEN and EP experiments. Culturing was done feeder-free on Geltrex coatings (#A1413302, Invitrogen) in Essential 8 medium (#A1517001, Invitrogen) supplemented with 1:100 Penicillin/Streptomycin (#15140-122, Invitrogen). Passaging of hESCs was done with TrypLE Select (#12563011, Invitrogen).

Prior to cell seeding, PEN cell culture substrates were coated overnight with 1:100 Geltrex on an orbital shaker platform. Next, 5×10⁴ hESCs were seeded on the PEN cell culture substrates. After 1 h of incubation at 37° C. in a humidified atmosphere with 5% CO₂ and 5% O₂, 1 mL of E8 Essential medium supplemented with 1:100 RevitaCell (A2644501, Invitrogen) was added to the 12 wells. After 24 h, the medium was replaced by Essential 8 medium and refreshed daily till the cell density achieved the required density in 3-4 days.

Before PEN photoporation, 0.5 mg/ml 10 kDa Alexa Fluor® 647 labelled dextran in cell medium was added to the cells. Post laser scanning at the indicated laser fluence, cells were further cultured for another 2 h before recording confocal microscopy images. Cell viability was determined by Cell Titer-Glo at the indicated times post treatment. Cell proliferation was quantified from confocal microscopy images as well as described below.

Electroporation using the P3 Primary Cell 4D-Nucleofector™ X Kit (Lonza, Cologne, Germany) with a Nucleofector™ 4D (Lonza, Cologne, Germany) was used to deliver 10 kDa Alexa Fluor® 647 labelled dextran, according the manufacture's protocols. In brief, 2×10⁵ single hESCs were re-suspended in the Nucleofector™ solution supplemented with a final concentration of 0.5 mg/ml Alexa Fluor® 647 labelled dextran. This solution containing cells was transferred to a 20 μL Nucleofector™ strip and electroporated using the indicated programs. For electroporation hESCs were detached with TrypLE, transferred to an electroporation cuvette and treated with the selected program (Costa, M. et al., Nat. Protoc., 2, 792-796 (2007); Helledie, T., Nurcombe, V. & Cool, S. M., Stem Cells Dev., 17, 837-848 (2008)).

After electroporation, the cells were washed with cell culture medium and transferred to a 48-well plate for further incubation at 37° C. Finally, delivery efficiency was quantified from confocal microscopy images and cell viability was measured by Cell Titer-Glo at the indicated times post treatment. Directed differentiation towards cardiomyocytes was done with the PSC Cardiomyocyte Differentiation Kit (#A2921201, Invitrogen) according to manufacturer's protocol. hESC and cardiomyocytes staining protocols were performed as follows. hESCs and cardiomyocytes were fixed for 20 min with 4% paraformaldehyde at RT. hESCs were permeabilized for 30 min with 0.1% Triton X-100 diluted in phosphate buffered saline (PBS). Subsequent incubation with blocking solution consisting of 5% Goat serum (#16210-064, Invitrogen) in PBS was done for 30 min. The cells were incubated overnight at 4° C. with primary antibodies diluted in PBS containing 0.05% Tween20 and 1% bovine serum albumin (BSA). The next day, cells were incubated for 30 min at RT with secondary antibodies diluted in PBS containing 0.05% Tween20 and 1% BSA and subsequently incubated for 10 min with 0.1% Hoechst solution (#H3570, Invitrogen). Immunostaining of CMs was performed with the exception that the primary antibody was incubated overnight at 4° C.

Single guide RNA targeting the IL-2R gamma gene (sequence: 5′-GGTAATGATGGCTTCAACA-3′) was purchased from Synthego. Cas9 RNP complexes were simply made by mixing either sgRNA in a 2.5:1 molar ratio with Cas9 endonuclease and allowing the complexes to assemble for at least 10 minutes at room temperature prior delivery. Extraction of genomic DNA was done using the innuPREP DNA Mini Kit (Analytik Jena, Jena, Germany) according the manufacturer's protocol. Genomic DNA of H9 stem cells was extracted using the InnuPREP DNA mini kit (Analytik Jena, Jena, Germany), according manufacturer's instructions. Next, a target DNA region in the IL-2R gamma gene was amplified using 100 ng genomic template DNA and the KAPA HiFi HotStart ReadyMix (Roche Diagnostics Belgium, Diegem, Belgium), and with forward primer 5′-ACCACCTTACAGCAGCACC-3′ and reverse primer 5′-ATGATGGTCAGAAGGAGGAGG-3′. PCR cycling conditions consisted of initial denaturation of 2 minutes at 98° C., followed by 35 cycles of denaturation at 98° C. (10 seconds), annealing at 65° C. (30 seconds), elongation at 72° C. (21 seconds), and a final elongation at 72° C. for 10 minutes. Amplified PCR products were purified using the by the QIAquick PCR purification kit (Qiagen, Chatsworth, CA, USA), according the manufacturer's protocol. The sequence of the PCR amplicons was eventually determined using Sanger sequencing by the GATC Lightrun service (Eurofins Genomics, Ebersberg, Germany) and using sequencing primer 5′-AGGACTTAGCCCGTGTC-3′. Knock-out levels were determined by Inference of CRISPR Edits (ICE) analysis (Synthego), using a nontreated sample as unedited control and assuring a model fit of R²>0.9.

### PEN Photoporation and Electroporation of Human CD3+ T Cells.

Human T cells were obtained from Ghent University hospital. Buffy coats were obtained from healthy donors after informed consent and approval. Peripheral blood mononuclear cells (PBMCs) were isolated via density centrifugation using Lymphoprep (Alere Technologies, Oslo, Norway). Next, PBMCs were incubated in IMDM (Gibco, Invitrogen, Merelbeke, Belgium) supplemented with 10% fetal calf serum ((FCS, Bovogen), 100 U/mL penicillin (Gibco, Invitrogen), 100 μg/mL streptomycin (Gibco, Invitrogen), 2 mM glutamine and 5 ng/ml IL-2 (Roche, Vilvoorde, Belgium) and stimulated with CD23/CD28 activator (Stemcell Technologies, Vancouver, Canada r) at a 1:1 bead to cell ratio. After 7 days the cells were harvested and re-incubated with X-ray irradiated (40 Gy) (SARRP) PBMCs (1:2 ratio) and X-ray irradiated (50 Gy) JY (5:1 ratio) feeder cells in complete IMDM supplemented with 1 μg/mL phytohemagglutinin (Remel Europe, KENT, UK). After an additional 14 days, CD3+ cells were harvested and used for experiments as further indicated. Feeder cells were irradiated using the Small Animal Radiation Research Platform (Xstrahl, Surrey, UK). For photoporation treatment, T cells were transferred to the culture substrates at a density of ˜8×10⁵ cells/well and already in the presence of the transfection molecules (if any). Cells were allowed to sediment on the fiber web for 5 min before starting the laser treatment.

CD70-specific CAR T cells were manufactured. Briefly, PBMCs were isolated via Lymphoprep and T cells were stimulated using Imunocult Human CD3/CD28/CD2 activator in complete IMDM supplemented with 10 ng/ml IL-12 (PeproTech, Hamburg, Germany). Cells were harvested 72 hours after stimulation and resuspended in retroviral supernatant. Next, cells were centrifuged for 90 min at 1000×g (32° C.) on retronectin coated plates (TaKaRa, Saint-Germain-en-Laye, France). Irradiated PBMCs (40 Gy) and irradiated JY cells (50 Gy) were used as allogenic feeder cells to expand transduced cells in completed IMDM supplemented with 1 g/mL phytohemagglutinin (PHA, Sigma-Aldrich). On day 5 and 10, 5 ng/ml IL-2 was added and every 7-14 days cells were restimulated. For photoporation treatment, CD3+ T-cells or CAR T-cells were transferred to the culture substrates at a density of ˜1.0×10⁶ cells/well and already in the presence of the transfection molecules. Cells were allowed to sediment on the fiber web for 5 min before starting the laser treatment.

FD10 kDa and siRNA were delivered in human T cells by electroporation using the P3 Primary Cell 4D-Nucleofector™ X kit (Lonza, Cologne, Germany) with a Nucleofector™ 4D (Lonza, Cologne, Germany), according the manufacture's protocol. In brief, 1×10⁶ CD3+ T cells or CAR T-cells were re-suspended in the Nucleofector™ solution supplemented with a final concentration of 2 mg/mL FD10 or 1 μM siRNA. The solution containing cells were transferred to 20 μL Nucleofector™ strip and electroporated using the program EO-100, EO-115 or FI-115. After electroporation, cells were washed with cell culture medium and transferred to a 96-well plate at 200 K cells per well for further incubation at 37° C. For siRNA transfection, viable human T cells were stimulated with Immunocult CD3/CD28 activator and 5 ng/ml IL-2 4 hours after treatment. After 24, 48 or 72 hours of incubation, cells were washed with PBS and analyzed using flow cytometry or confocal microscopy as indicated.

siRNA Transfection and PD1 Expression Analysis of Transfected T Cells.

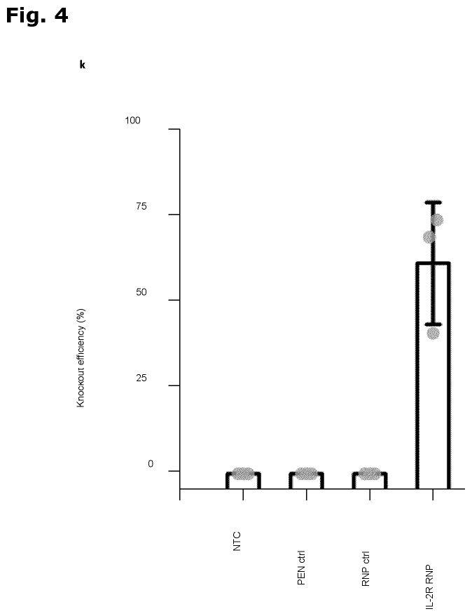

For siRNA transfections of human T cells, siRNA duplexes targeting programmed cell death protein 1 (PD-1) and negative control duplexes (siCTRL) were ordered from various manufacturers (FIG. 8). Human T cells were PEN photoporated or electroporated as previously described, in the presence of the indicated concentration of siRNA. After treatment cells were washed twice with PBS and resuspended in complete IMDM at 2×10⁵ cells per well in a 96-well plate (#10062-900, VWR®, US). After 4 hours, human T cells were stimulated with Immunocult Human CD3/CD28 activator (Stemcell Technologies, Vancouver, Canada) and 5 ng/ml IL-2 to upregulate PD1 expression unless otherwise specified (e.g. unstimulated condition). At the indicated timepoints PD1 expression was evaluated using flow cytometry. Briefly, human T cells were washed with PBS and re-suspended in FACS buffer. Next, T cells were incubated with PD1PE (Milteny Biotec, Germany) for 30 min at 4° C. after which the cells were washed and incubated for 10 min with TO-PRO™-3-iodide. The data obtained from flow cytometery was post-processed with the FlowJo software package (Treestar Inc, Ashland, USA). TO-PRO™-3 iodide (APC channel) was used to exclude dead cells from further flow cytometry analysis. Knockdown efficiency of PD1 expression was calculated according to equation (6):

$$Knockdown efficiency ( \% ) = 100 \% - ( \frac{{MFI}_{sample} - MFIUnstimulated}{{MFI}_{siCTRL} - MFIUnstimulated} ) \times 1 \; 00 \% \qquad ( 6 )$$

Here, MFIₛₐₘₚₗₑ is the mean fluorescence intensity of cells treated with PD1 siRNA; MFI_{unstimulated} is the mean fluorescence intensity of unstimulated T cells under identical experimental conditions; MFI_{siCTRL} is mean fluorescence intensity of cells treated with negative control siRNA.

### Characterization of T Cell Phenotype and Intracellular Ca²⁺ Analysis.

The following antibodies were used for flow cytometry analysis of human CD3+ T cells: CD3 BV421 (Pacific blue, Invitrogen, Belgium), CD4 BB700 (PERCP-Cy5.5, Invitrogen, Belgium), CD8 APC-Cy7 (Invitrogen, Belgium), CD137 PE (Biolegend, USA), CD154 FITC (Biolegend, USA), PD1APC (Invitrogen, Belgium) and PD1PE (Milteny Biotec, Germany). Briefly, T cells were washed with PBS (PBS, Gibco-invitrogen) and re-suspended in FACS buffer, supplemented with 5% bovine serum albumin (BSA, Sigma-Aldrich, Bornem, Belgium). After 30 min incubation at 4° C. with the indicated antibodies, the cells were washed and analyzed by flow cytometry. Non-treated cells were used to set the 90% threshold value above which cells are considered positive %. Intracellular Ca²⁺ was measured using a Fluo-4 Direct™ Calcium Assay Kit (#F10471, Invitrogen) according to the manufacturer's instructions.

### Analysis of T Cell Proliferation Using Confocal Microscopy

After PEN photoporation or electroporation, using optimized delivery protocols (see main text), T cells were washed twice and seeded at 2×10⁵ cells per well in a 96 well plate. After 4 hours T cells were stimulated with 5 ng/ml IL-2 and Immunocult human CD3/CD28 activator in complete IMDM. At the indicated timepoints T cells were washed and stained with Calcein AM and TO-PRO-3 iodide for 30 min in cell medium. Living cells were detected and quantified based on their green (Calcein AM positive, living cells) and red (TO-PRO-3 negative, dead cells) fluorescence levels using an A1R confocal microscope (Nikon, Badhoevedorp, The Netherlands) equipped with a perfect focus system and a ×20 objective lens (CFI Plan Apochromat, Nikon, Badhoevedorp, The Netherlands). The software package ImageJ with the plugin of Analyze Particles was used for image processing.

### Cytokine Expression Analysis of Human T Cells.

To analyze the cytokine secretion profile of electroporated or PEN-photoporated T cells, human T cells were seeded in a 96-well plate at 1×10⁶ cells per well for up to 48 hours post-treatment. At the indicated time-points, supernatant was collected for cytokine secretion analysis. Cytokine secretion of 10 different cytokines, including IL-5, IL-6, IL-9, IL-10, IL-13, IL-17A, IFN-γ and TNF-α, was quantified using a multiplex bead assay (LEGENDplex, Biolegend) according to the manufacturer's instructions.

### ⁵¹Chromium Release Cytotoxic Killing Assay.

Cytotoxic killing of CAR transduced T cells exposed to electroporation or PEN photoporation (without one or more (macro) molecules otherwise not present in a native T cell) was measured using a ⁵¹Chromium release assay as previous described⁵⁰. Both SKOV3 and H1650 cells were used as target cancer cell lines. CD70-specific CAR T cells were PEN photoporated, electroporated or left untreated, as previously described, followed by 48 hours of culturing in complete IMDM supplemented with 5 ng/ml IL-2. Target cells were labeled with ⁵¹Chromium (Perkin Elmer, Zaventem, Belgium) for 90 min at 37° C. After several washing steps, 10³ target cells were added per well in a 96 well V-bottom plates (NUNC, Thermo Fisher Scientific, Merelbeke, Belgium). Various amounts of CAR T cells were added at the indicated effector-target cell ratios. Next, supernatans was collected 4 hours later and measured in a 1450 LSC & Luminescence Counter (Perkin Elmer, Zaventem, Belgium). Specific lysis was calculated using the following formula: (experimental release-negative control release)/(positive control release-negative control release)×100%. Here, negative control release is the release induced by only target cells in regular cell culture medium; positive control release is the release of the complete lysis of the target cells by adding 2% tritonis in cell culture medium; experimental release is the release of the samples under coordinate experimental conditions.

### CAR-T Treatment of SKOV3 Mouse Tumor Model.

Buffy coats from healthy donors were obtained from the Belgian Red Cross and used following the guidelines of the Medical Ethical Committee of Ghent University Hospital, after informed consent had been obtained, in accordance with the Declaration of Helsinki. PBMCs were isolated by Lymphrop (StemCell Technologies) gradient centrifugation. The percentage of CD3+ cells was determined by flow cytometry and T cells were stimulated with Immunocult Human CD3/CD28/CD2 T cell activator (StemCell Technologies) according to the manufacturer's instructions. Cells were harvested 48 hours after stimulation, resuspended in retroviral supernatant and centrifuged on retronectin (TaKaRa) coated plates. Two days after transduction, cells were harvested and cultured for 8 days in the presence of 10 ng ml-1 IL7 and IL15 (Miltenyi). At day 11 post stimulation, CAR T cells were harvested, washed using sterile PBS and diluted in PBS for intravenous injection in mice. The expressed CAR is composed of an anti-hCD70 VHH, a CD8α-based hinge, the co-stimulatory domain of 4-1BB (CD137), and the T-cell receptor-derived signaling domain CD3ζ.

NSG mice were subcutaneously injected with 2×10⁶ SKOV3 cells. When tumors reached a size of 4-7 mm in diameter, mice were injected intravenously with PBS or 5×10⁶ non-transfected or transfected CAR T cells with either the transfected or the nontreatment. The next day, mice were injected intraperitoneally either with PBS or with 100 μg Nivolumab (Opdivo, Bristol Myers Squibb). Tumor size was measured with a caliper.

### Statistical Analysis.

Differences between two datasets were assessed using one-way ANOVA and multiple comparisons were adjusted by Bonferroni corrections. Statistical significance is indicated as follow: *P<0.05, **P<0.01, ***P<0.001, ****P<0.0001.

### Results

### Synthesis and Characterization of Photothermal Electrospun Nanofibers (PENs).

Nanofibers were prepared from a mixture of polycaprolactone (PCL) and iron oxide nanoparticles (IONPs) dissolved at various weight percentages in a N,N-Dimethylformamide (DMF)/Tetrahydrofuran (THF) solution. Fibers were collected on microscope glass slides as shown in FIG. 1a,b. Scanning electron microscopy (SEM) and transmission electron microscopy (TEM) revealed an average fiber diameter of ˜300 nm irrespective of the IONP concentration (FIG. 1b-d). By confocal microscopy it was found that the PEN web thickness gradually increased up to 4 μm after 1 h of electrospinning (FIG. 1e,f). As the webs did not change much after 30 min, we selected this electrospinning time for all fiber webs created from here on. When adding increasing amounts of IONPs to the nanofibers, the PEN web's thickness did not change significantly (FIG. 1g).

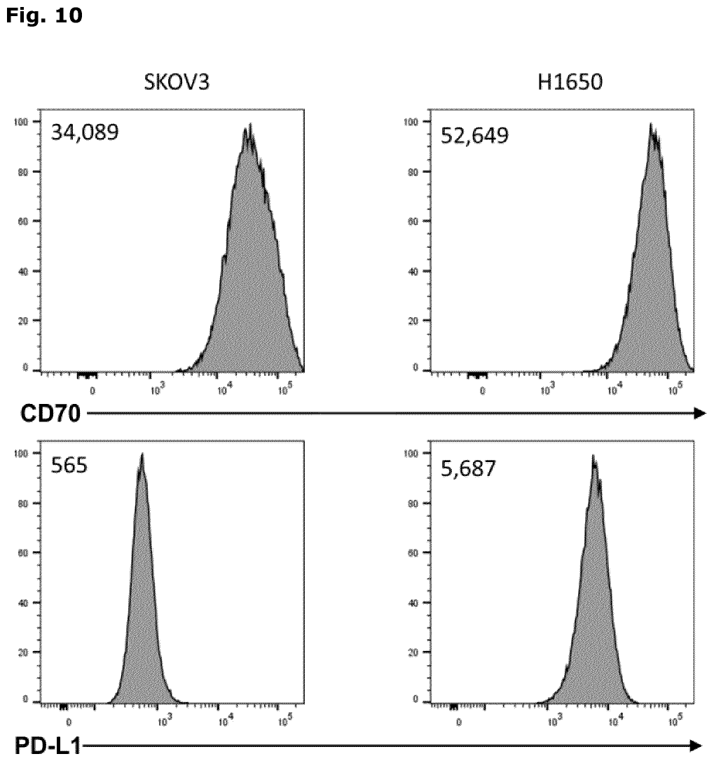

Next, we analyzed how IONPs are distributed within the nanofibers. While IONPs were difficult to see by SEM when operated at 1.5 kV, they could be clearly seen when increasing the voltage to 20 kV (FIG. 1h). Thus, we found that the IONP density linearly increased from 1.7 to 192 clusters/1000 μm² for 0.02% to 5% IONPs (FIG. 1i). Two dimensionless size parameters allow to further understand the IONP distribution. The first is \tilde{l}₁ (FIG. 12j), defined as the ratio of the apparent IONP cluster size d_{c} over the average diameter of a single IONP dₛ (162±41 nm as quantified by TEM images, see FIG. 12c). If \tilde{l}₁>1 IONPs are in a clustered state, examples of which are shown in FIG. 1k. For a PEN web with 1% IONPs, we observed that \tilde{l}₁≥2 for more than 90% of IONPs clusters in the fibers, indicating that most of the IONPs are present in a clustered state. The second dimensionless size parameter is \tilde{l}₂ (FIG. 1j), defined as the ratio of the IONP cluster size d_{c} and the nanofiber diameter D. We found that >80% of the IONPs clusters occupied more than half of the nanofibers diameter. Examples are shown at the top of FIG. 1I. Finally, we analyzed the distance h that IONP clusters are below the fiber surface. If h>0 nm, the cluster is below the fiber surface, if h=0 nm the cluster surface coincides with the fiber surface, and if h<0 nm, the cluster is sticking out of the fiber. Examples are shown at the top of FIG. 1m. As 86% of the IONPs clusters were less than 40 nm away from the fiber surface, one can expect excellent heat transfer from the IONPs to the fiber surface which will be in contact with the cell membrane, as will be examined in detail further on. It is of note that, although there is a small fraction of IONPs sticking out of the fiber (˜10%, h<0 nm), they are still covered by a very thin layer of PCL polymer, as can be seen in high resolution TEM images (FIG. 12d). We did not observe substantial differences for any of those three parameters when increasing the IONPs content from 1% to 5% (FIG. 12e).

### PEN Photoporation Enables Safe and Efficient Intracellular Delivery in Adherent Cells.

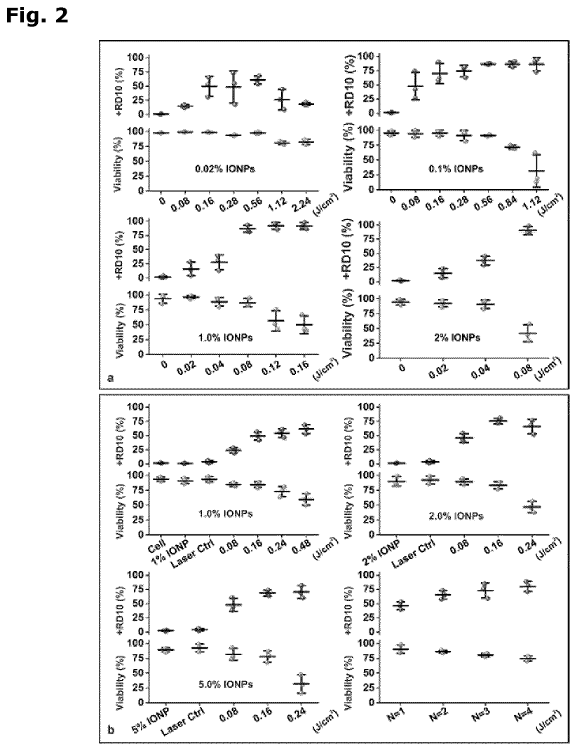

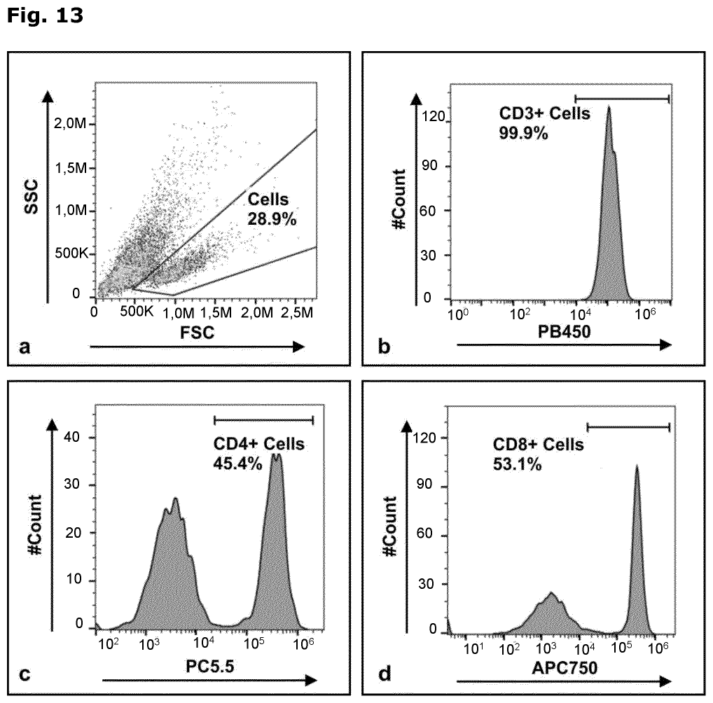

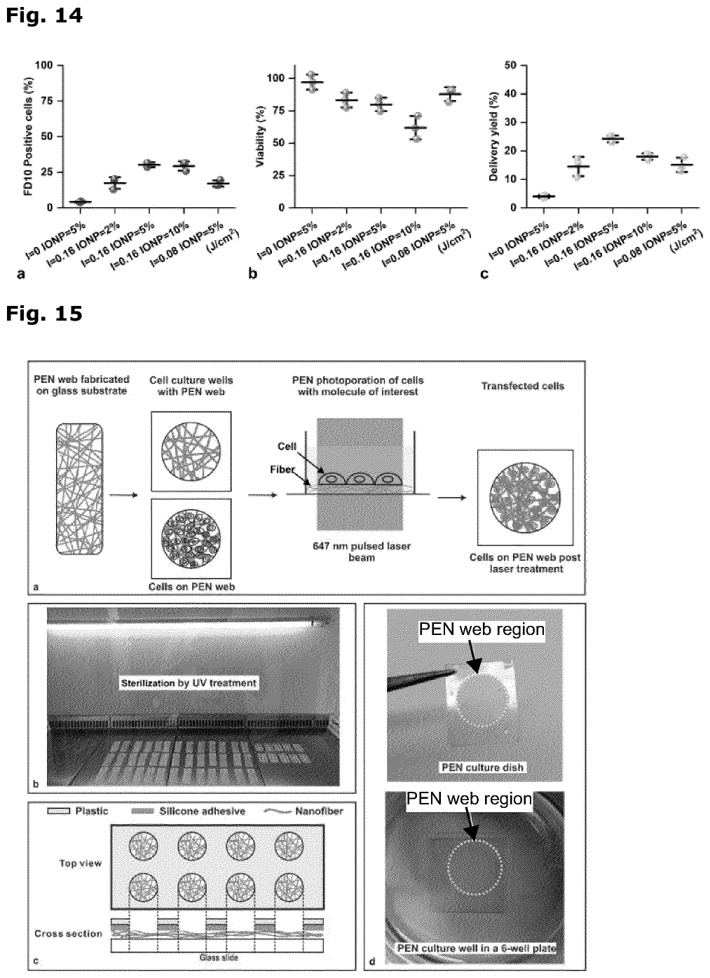

Intracellular delivery by PEN photoporation was initially tested on Hela cells. Cell culture wells were prepared from PEN webs as illustrated in FIG. 15. Fibers were coated with collagen to facilitate cell attachment (FIG. 16a), which substantially increased the cell density and cell area as compared to cells grown on bare PCL fibers (FIG. 16b, c). Depending on the IONP content the average number of IONP clusters per cell ranged from 0.26 (0.02%) to 159 (5.0%) (FIG. 16d). To test successful intracellular delivery by PEN photoporation, red fluorescently labelled dextran of 10 kDa (RD10) was added to the cells cultured in PEN wells. After scanning one time with the 7 ns pulsed laser beam, cells were washed and the Calcein AM viability stain was added to the cells. Exemplary confocal images are shown in FIG. 16e showing increasing intracellular delivery of RD10 with increasing laser fluence. Quantification of confocal images revealed that increasing the laser fluence or IONPs content generally lead to more intracellular delivery, although cell toxicity gradually increased as well (FIG. 2a). We opted to continue working with 1% IONP PEN webs and a laser fluence of 0.08 J/cm² as this resulted in optimal delivery efficiency with the least amount of laser energy.

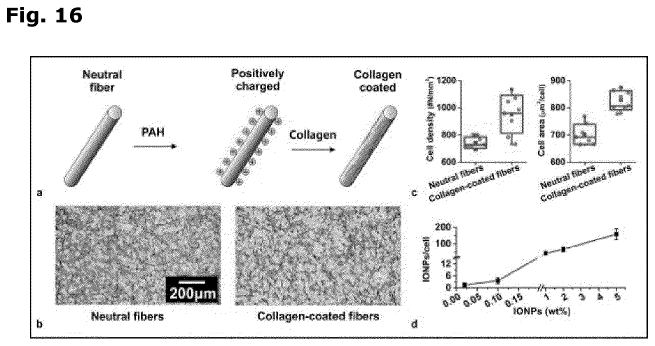

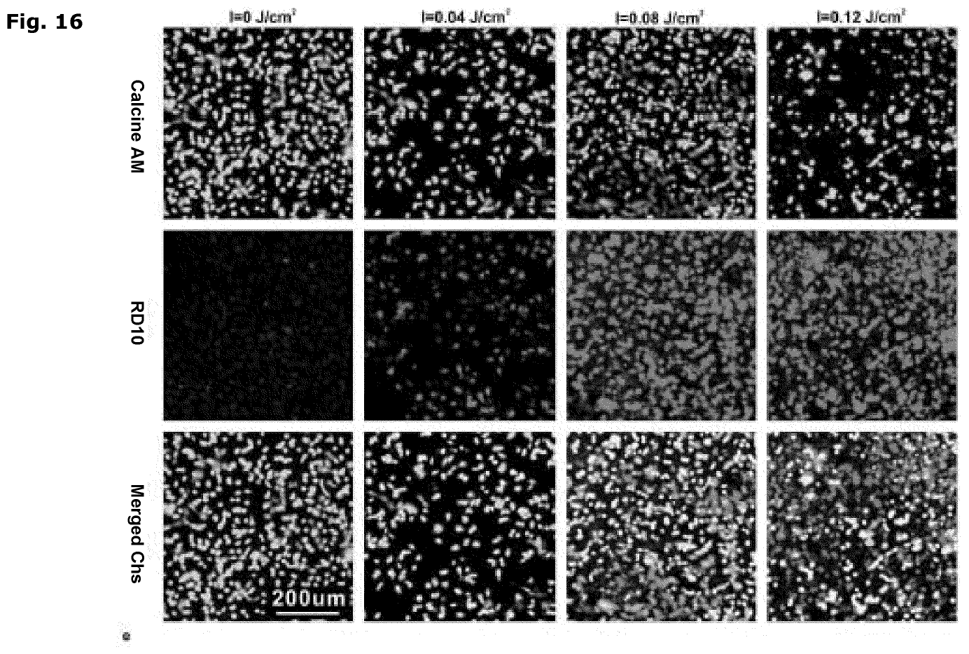

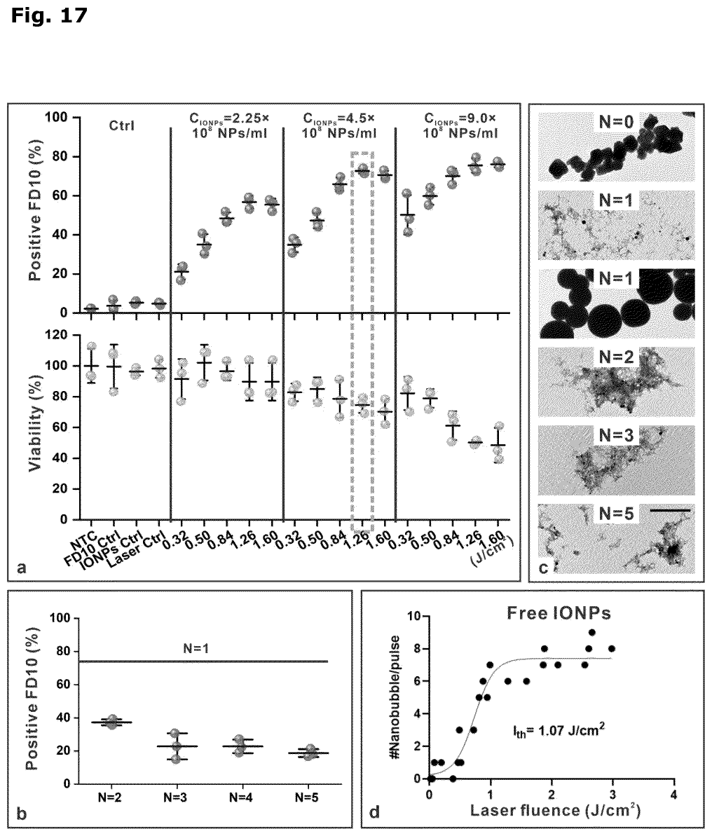

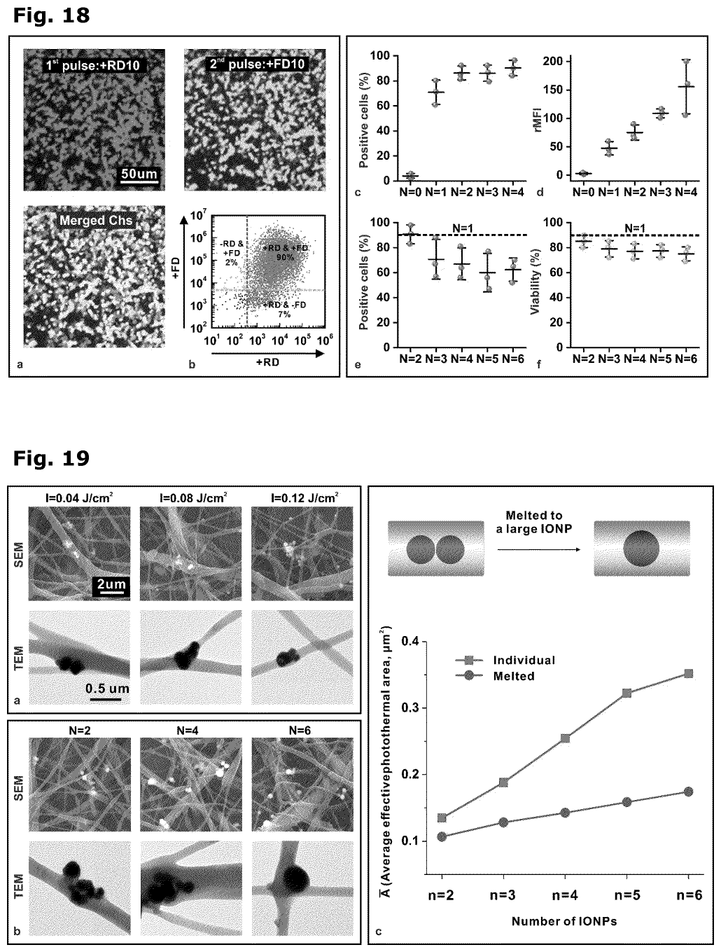

Traditional NP-sensitized photoporation makes use of gold NPs which can be activated only once because they tend to fragment after already the first laser pulse, resulting in a loss of their photothermal functionality. We found that the same holds true for photoporation with free IONPs, in which case optimal delivery efficiency was obtained at a laser pulse fluence of 1.26 J/cm² (FIG. 17a). Clearly the free IONPs lost their activity already after the first laser pulse (FIG. 17b). At such high laser fluences vapor nanobubbles are formed (FIG. 17d), resulting in particle fragmentation (FIG. 17c). Considering that much lower laser fluences were sufficient to obtain optimal delivery with PEN webs in which IONPs are imbedded into PCL nanofibers, it is of interest to see if the PEN webs can perhaps be activated multiple times by repeated scanning of the laser beam. We hypothesized that this may be possible since the sensitizing particles are stabilized by the surrounding polymer material and are irradiated with less than 1/10th of the laser fluence that is typically used in traditional photoporation. We started by irradiating cells on a PEN substrate two times in a row. In the first round of PEN photoporation we delivered RD10 as before, after which the cells were washed and irradiated a second time on the same PEN substrate but now in the presence of 10 kDa green fluorescent FITC-dextran macromolecules (FD10). When examining the cells by confocal microscopy afterwards, it became clear that most cells had both red and green fluorescence (FIG. 18a). It can be noticed that cells exhibiting strong green fluorescence do not necessarily have strong red fluorescence, and vice versa. This is likely due to the fact that there is about 10 min time between both experiments, which is the time needed to do the first photoporation treatment followed by washing of cells, adding the new dextrans and starting the second photoporation procedure. During that time cells might slightly move or reshape their cell surface so that they make contact with the fibers at different places, likely contributing to some variability in the delivery efficiency between both photoporation treatments. Nevertheless, quantitative analysis by flow cytometry confirmed that 90% of cells were positive for both RD10 as FD10 (FIG. 18b). To provide further evidence of repeated photoporation with the same PEN webs, we continued photoporating Hela cells up to 4 times with FD10. The FD10 concentration was doubled (from 0.2 mg/ml to 1.6 mg/mL) between each photoporation round to more easily see the increase in intracellular delivery (which is still diffusion driven). While the percentage of positive cells increased from ˜70% to ˜90% (FIG. 18c), the increased delivery was most apparent from the relative mean fluorescence per cell (rMFI) which increased almost linearly with each round of photoporation (FIG. 18d). 5 Having established that PEN webs can be repeatedly activated, we wanted to determine more precisely to which extent PENs web lose photoporation capacity with each additional scan. To this end we again performed repeated PEN photoporation, but now only added FD10 to the cells just before the last scan. For instance, in case of N=4 scans, the first three laser scans are performed with the cells still in normal culture medium (without FD10), while FD10 is added to cell medium prior to the fourth and last scan. When looking at the percentage of transfected cells, a small drop in PEN substrate functionality is observed when photoporating more than 2 times. However, the data clearly showed that the PEN web can be activated for at least 6 times, still transfecting 60-70% of the cells (FIG. 18e). Interestingly, repeated photoporation of the cells did only have little effect on cell toxicity, with 75% viable cells even after 6 times PEN photoporation (FIG. 18f). The slight reduction in photoporation efficiency upon repeated activation may be due to either morphological changes happening to the IONPs (clusters) or possibly a release of (a part of) the IONPs from the fibers. Since the absence of IONPs release is a fundamental premise in our study, it will be investigated and discussed separately below. At this point we can already confirm that IONPs release does not happen, so that we here focus on potential morphological changes of the embedded IONPs after (repeated) laser irradiation which was investigated by SEM and TEM. As SEM and TEM images in FIG. 19a show, IONPs remained unchanged after a single laser scan at the lowest fluence of 0.04 J/cm2. At higher fluences the IONPs tended to melt and form larger spherical structures probably due to the high temperature that is reached. For instance, at a laser pulse fluence of 0.12 J/cm² a temperature of ˜1800° C. is reached in the particles (see simulations further on), which is already above the iron oxide melting temperature (1565° C.). A similar phenomenon was observed when exposing the PEN webs to multiple laser scans (0.08 J/cm²), finding that gradually more and more IONPs clusters reshaped into larger spherical particles (FIG. 19b). Since this reduces the effective photothermal area to some extent, this may explain why the photoporation efficiency dropped slightly upon repeated laser activation (FIG. 19c). Until now we evaluated intracellular delivery of a 10 kDa model marker, which is of a similar size as for instance antisense oligonucleotides or siRNA. In addition, it is of interest to evaluate to which extent larger macromolecules can be delivered, with a molecular weight closer to that of proteins or mRNA. To that end we used 40 kDa, 70 kDa, 150 kDa and 500 kDa FITC-dextran (FD40, FD70, FD150 & FD500) as model molecules, which were delivered in Hela cells by 1×, 2× and 4×PEN photoporation. As shown in FIG. 20a, b, delivery efficiency gradually decreased for increasing molecular weight, which is due to a combination of molecules becoming large compared to the pore size as well as slower molecular diffusion. Repeating the photoporation procedure generally resulted in slightly more positive cells, while it did not improve the amount delivered per cell. Still, we conclude that PEN 6 photoporationis successful in transfecting cells with compounds up to at least 500 kDa, with a very substantial 65-90% transfected cells depending on the molecular size.

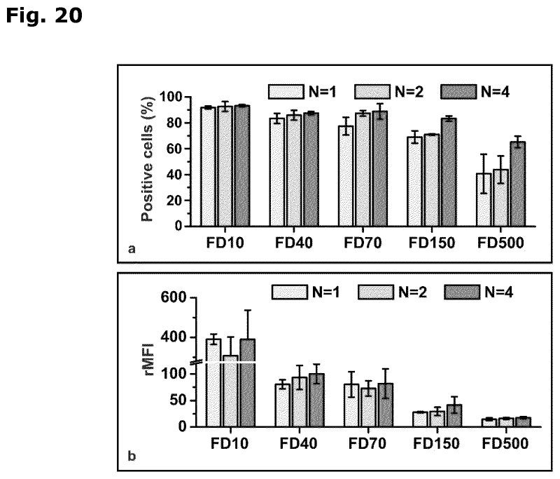

As explained in detail in above, we found that, contrary to free IONPs (FIG. 17), PEN substrates can be repeatedly laser-activated, leading to gradually enhanced delivery efficiency (FIG. 18, 19). This proved to be most useful for the delivery of large macromolecules (FIG. 21).

### Efficient Intracellular Delivery in Suspension Cells by PEN Photoporation.

Next we tested if PEN photoporation can be used to deliver compounds in suspension cells. For this we used Jurkat cells, which is an immortalized line of human T lymphocytes and a widely used model for hard-to-transfect primary human T cells. Cells were added in the presence of 10 kDa FITC-dextran (FD10) to PEN culture wells and allowed to sediment on the fibers for 5 min (FIG. 21a). Depending on the IONP content, the number of IONP per cell ranged from 7.7 to 28.4 IONPs/cell (FIG. 21b). Initial delivery experiments showed that positively charged nanofibers produced the best results rather than collagen coated ones (FIG. 21c). From image analysis it was determined that the delivery efficiency increased with increasing laser fluence or IONP content at the expense of cell viability as measured by the calcein red-orange AM viability stain (FIG. 2b). If we set a threshold of minimal 80% viability, the best transfection efficiency (˜75% positive cells) was obtained for a PEN substrate with 2% IONPs (˜12 IONPs/cell, FIG. 21) and a laser fluence of 0.16 J/cm². Finally, we again tested repeated PEN photoporation (bottom left panel FIG. 1b), finding that the percentage of positive cells could be increased by repeating the procedure with only little effect on cell viability. Note that for this experiment we used a PEN substrate with 2% IONPs with a suboptimal laser fluence of 0.08 J/cm² to better show the gradual improvement.

ICP-MS/MS Confirms there is No Leakage of IONPs from PEN Substrates Upon Laser Irradiation.

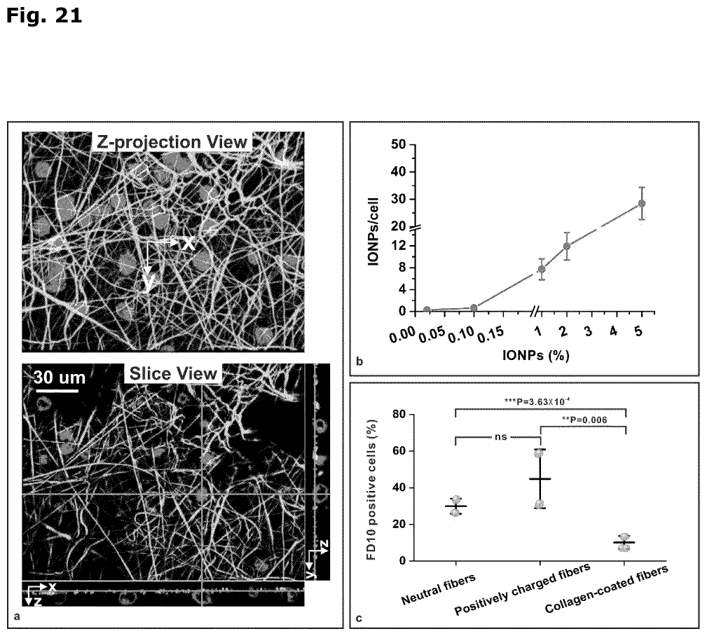

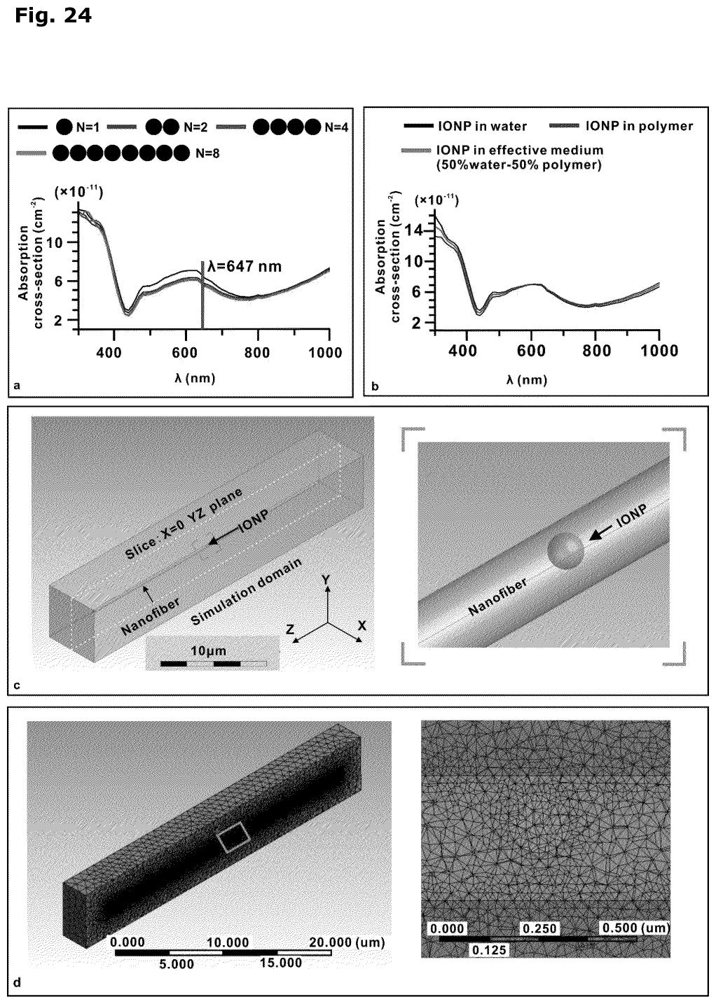

A crucial premise in this work was to avoid direct contact between sensitizing NPS and cells during photoporation. To verify whether condition is met, the cellular iron concentrations were determined using ICP-MS/MS (tandem ICP-mass spectrometry) after PEN photoporation. Hela and Jurkat cells were photoporated using PEN substrates containing 1% or 2% IONPs, respectively. Next, as schematically shown in FIG. 2c, the cells were detached from the nanofibers and digested with aqua regia (3:1 mixture of hydrochloric acid and nitric acid) prior to ICP-MS/MS analysis. As a positive control, we included cells incubated with 500 μg/ml of 30 nm IONPs coated with polyethylene glycol for 4 h at 37° C. As shown in FIG. 2d, the positive control indeed had a significantly higher iron concentration in comparison with the negative control (untreated cells) for both cell types. Importantly, however, the iron content in PEN photoporated cells did not differ significantly from untreated cells irrespective of the laser fluence or number of laser scans. While this proves that there is no measurable increase in iron content in cells, one could argue that the endogenous iron content in cells is already fairly high so that small increases may not be easily detected. Therefore, we proceeded with measuring potential iron release from the PEN substrates when submerged in pure DI water (without any cells present, FIG. 2e). The iron content in DI water after laser activation of the PEN substrates did not significantly increase and remained below the detection limit of 0.08 mg/L irrespective of the IONP content, number of scans or laser fluence (FIG. 2f). Instead, when IONP were intentionally released by digestion of PEN fibers with aqua regia, very high iron concentrations proportional to the embedded IONPs content (1, 2 or 5% IONPs) were indeed measured. All together we it can be concluded that IONP are not released from PEN substrates upon laser activation, thus avoiding any direct exposure of cells to potentially toxic sensitizing NPs or its constituents. Detailed numerical simulations show that efficient cell permeabilization is nevertheless possible by the fact that IONPs are close to the fiber surface, allowing efficient heat transfer towards distinct places of the cell membrane where pores are formed (FIG. 22-26).

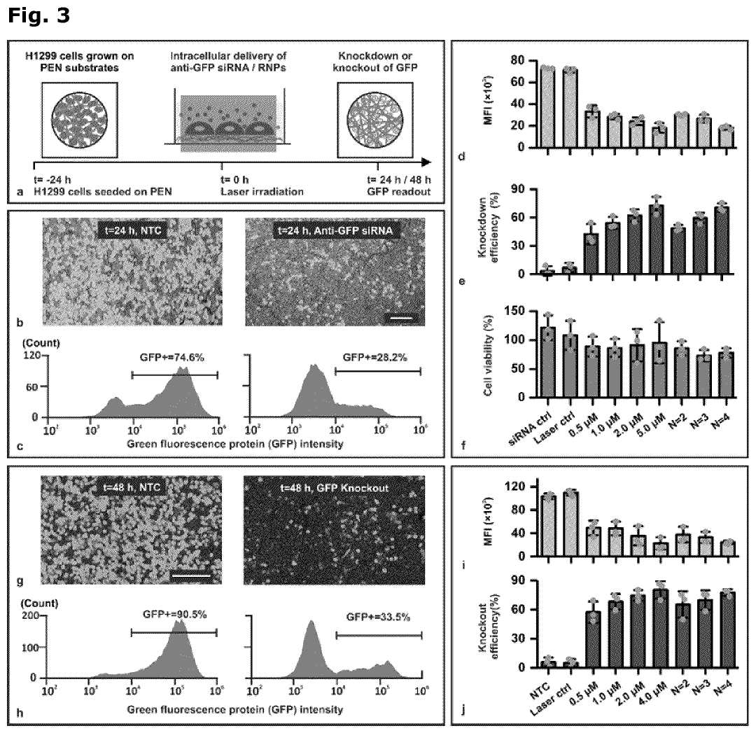

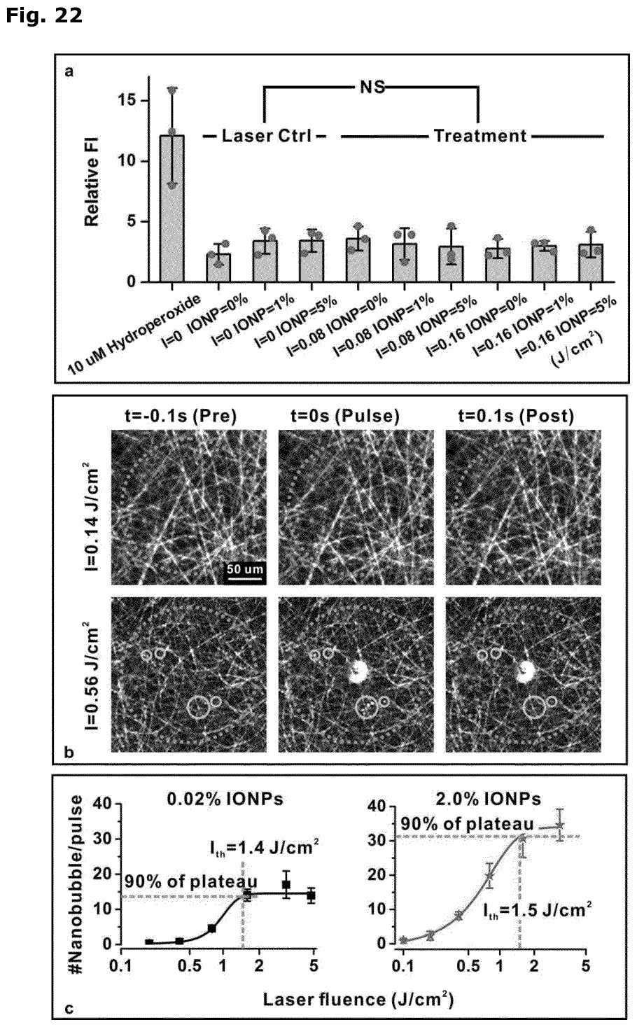

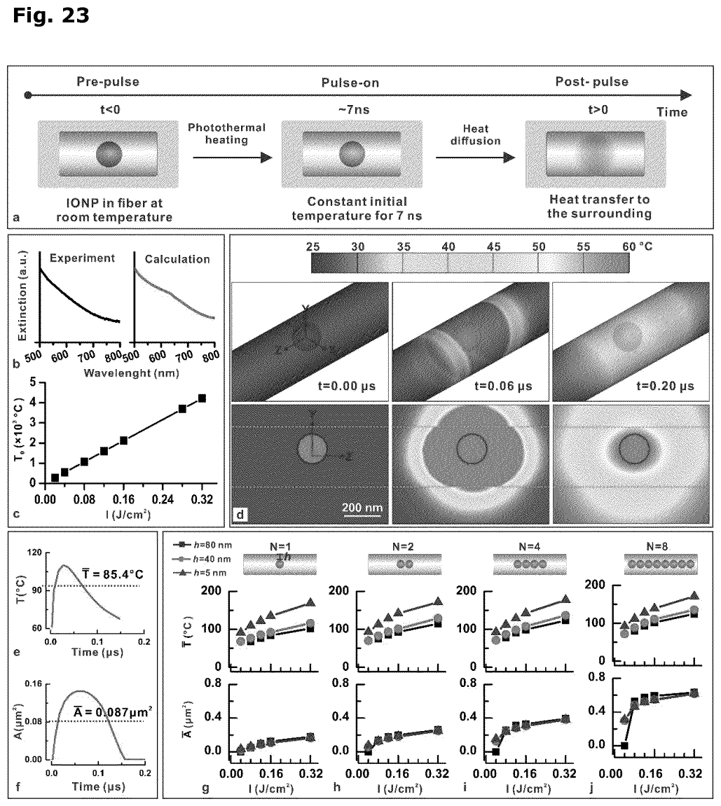

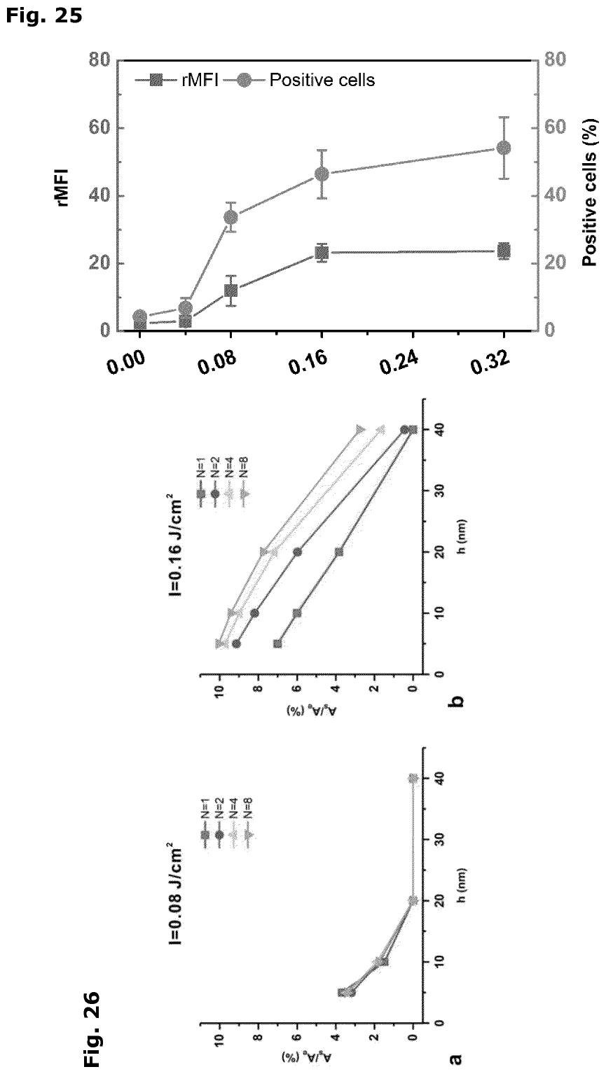

Given these positive results on adherent and suspension cells and having confirmed that the delivery process happens in a NPs-free manner, we continued investigating into greater details of the mechanism behind the cell membrane permeabilization by PEN photoporation. In traditional NPssensitized photoporation, membrane permeability can be induced by photothermal effects (heat or mechanical energy) or a photochemical process1. Photochemical processes include the generation of reactive oxygen species (ROS) which primarily occurs when irradiating the sensitizing NPs with ultrafast femtosecond or picosecond pulsed lasers 2, 3, 4, 5. Since in this work we used a much larger pulse width (7 ns), it seemed unlikely that permeabilization is caused by photochemical processes. Indeed, using 2′, 7′-Dichlorofluorescin (DCFH) as a fluorescent ROS indicator, we found no noticeable ROS after laser irradiation of PEN webs with IONPs content of up to 5% and laser fluence up to 0.16 J/cm² (FIG. 22a). This means that a photothermal mechanism must be responsible for the cell membrane permeabilization, which can be either mechanical damage through the formation of vapor nanobubbles or thermal damage through direct heating of the cell membrane. We investigated the potential formation of VNBs with dark field microscopy by which VNBs can be easily visualized as reported before by us and others6, 7. Indeed, VNBs are visible in dark field images as brief localized bursts of light. When using a relatively low laser fluence of 0.14 J/cm2, which is similar to the optimal condition for Hela's (0.08 J/cm²) and Jurkats (0.16 J/cm²), no VNB could be observed (FIG. 22b). Only at substantially higher laser fluences (>0.5 J/cm²) VNBs started to appear, similar to what we observed for free IONPs (FIG. 4d). By counting the number of VNBs within the laser irradiation area for increasing laser fluence, one can determine the VNB threshold, defined as the laser pulse fluence at which 90% of the plateau of producing VNB. The VNB generation threshold was virtually identical for PEN webs with 0.02% and 2% IONPs with a value of 1.4-1.5 J/cm2 (FIG. 22c). This is about 10× higher than the highest laser fluence used for PEN photoporation, so that we can safely exclude VNBs formation as the dominant permeabilization mechanism. This leaves a pure heating mechanism as the only left plausible mechanism for membrane permeabilization. It is of note that it cannot be simple bulk heating, since fibers with 5% IONPs irradiated by a single laser pulse of 0.16 J/cm2 can only increase the bulk temperature by 0.005 K (see ‘Methods’ for details on the calculation). Therefore, what most likely happens is that the temperature 7 is rapidly and locally increased at the IONPs (cluster) sites within the fibers, which permeabilize locally the cell membrane where it is in contact with the fiber hot spots. As it is virtually impossible to investigate this experimentally due to the extremely short time and small spatial scales, we investigated this more insight with theoretical calculations and numerical simulations of heat transfer from IONPs to the surrounding following the absorption of a laser pulse, as schematically shown in FIG. 23a. From the Generalized Multiparticle Mie Theory simulations the optical properties of the IONPs we calculated absorption cross-section which was further used to calculate the initial IONPs temperature and numerically solved the heat transfer differential equations in 3D to simulate heat transport from the IONPs into the fibers and surrounding cell medium. Note that potential phase changes were neglected during heat diffusion, which seems a reasonable assumption in the absence of VNBs generation. The calculated UV-VIS extinction spectrum as calculated from Mie Theory matched well with the experimental spectrum of IONPs dispersed in DI water (FIG. 23b). Since we have already shown that IONPs tend to be present in clusters within the fibers, we calculated the absorption cross section spectra for sets of IONPs in close proximity. As can be seen in FIG. 24a, this does not have a great impact on the value of the absorption cross section at 647 nm, probably due to only weak electromagnetic coupling between individual IONPs. We also calculated the absorption cross section spectra for IONPs in different media (PCL polymer, water, or an average of both), again finding that it does not appreciably change the value of the absorption cross section at 647 nm (FIG. 24b). As such we used the theoretical absorption cross section at 647 nm of a single IONP to calculate its initial temperature (TO) upon absorption of a single 7 ns laser pulse (λ=647 nm) for various laser fluences. As shown in FIG. 23c, initial temperatures could easily exceed the iron oxide melting temperature of 1565° C. For instance, already at a laser fluence of 0.12 J/cm² a temperature of ˜1600° C. is reached. Next, a 3D model was built to simulate the heat transfer of a single 160 nm IONP to the surrounding environment, consisting of a PCL fiber of 320 nm diameter and surrounded by water (FIG. 24c). Simulations were performed for the absorption of a 0.08 J/cm² laser pulse by an IONP that is separated by a distance h=40 nm from the fiber surface (cfr. FIG. 1m). Since the duration of a single pulse is 7 ns, we started the simulations by setting the IONP temperature to 1069° C. for this duration. The subsequent heat transfer is shown in FIG. 23d where temperatures >60° C. are color-coded in red, which is reportedly the temperature at which cell membranes become completely permeable 8, 9, 10. As can be seen, a substantial area at the fiber surface reaches >60° C., although within a very short time interval of tens of nanoseconds only. This area is analyzed as a function of time for the upper side of the fiber. In FIG. 23e, f the time course of A is plotted, which represents the size of the areas >60° C., together with the average 8 temperatures T over these areas. The average temperature remains >60° C. for a time period of 137 ns, reaching a maximum of 110.1° C. after 27 ns and with a time average value T=85.4° C. The area >60° C. is on average A⁻=0.087 μm², with a maximum size of 0.145 μm² which is reached after 57 ns. Next, we investigated how T and A⁻ depend on the laser pulse fluence (I=0.04-0.32 J/cm2), the number of locally clustered IONPs (N=1, 2, 4, 8) and the distance h (5, 40, 80 nm) from the fiber surface. Note that a maximum of h=80 nm was chosen since more than 90% IONPs had h277° C. is reached, we performed additional calculations for IONP at various depths below the fiber surface (h=5, 10, 20 and 40 nm). Simulations were performed for a laser pulse of 0.08 J/cm² or 0.16 J/cm², which are the optimal settings for adherent and suspension cells, respectively. In addition, we repeated these calculations for clusters of IONP consisting of 1 to 8 individual nanoparticles (N=1, 2, 4 and 8). From these simulations we calculated the ratio of the fiber surface area that reaches a temperature above 277° C. 9 (As) and above 60° C. (Ae). The ratio As/Ae indicates the relative importance of membrane pore formation by vapour nanobubbles (T>277° C.) or by direct heating (T>60° C.). FIG. 26 shows that for a laser fluence of 0.08 J/cm² As/Ae is less than 5% in all cases, and even is 0% when h≥20 nm. For a laser fluence of 0.16 J/cm² As/Ae is less than 10% in all cases. Together these theoretical considerations show that, even though bubble formation cannot be fully excluded, under the conditions used in our work it is quite unlikely to take place, or at least is not a substantial contributor to the cell permeabilization process which is almost purely heat mediated. These calculations are further supported by dark field microscopy experiments in which vapor nanobubble generation was not observed under similar conditions (FIG. 22b, c).

### Efficient Gene Silencing or Knockout in Adherent Cells by PEN Photoporation.

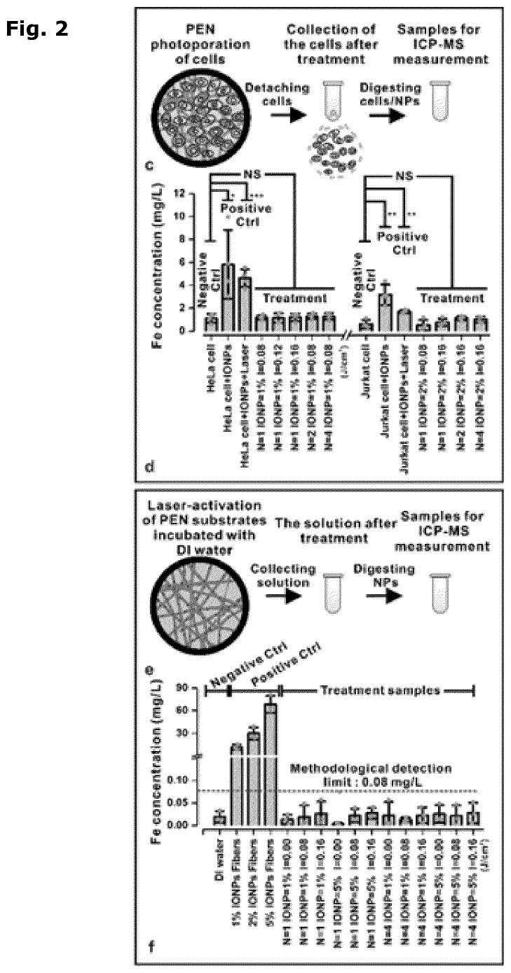

After successful delivery of model macromolecules, we went on to test delivery of SIRNA as a functional macromolecule, starting by delivering anti-GFP siRNA into adherent H1299 cells which stably express green fluorescence protein (GFP). As illustrated in FIG. 3a, cells were grown on collagen-coated PEN webs (1% IONPs) at 37° C. for 24 h, after which they were PEN photoporated (0.08 J/cm²) with control and anti-GFP siRNA. Confocal microscopy confirmed qualitatively successful siRNA knockdown after 24 h when using 5 μM siRNA (FIG. 3b), which was confirmed quantitatively by flow cytometry (FIG. 3c). Knockdown efficiency increased with SIRNA concentration (0.5, 1, 2 and 5 μM) without affecting cell viability, here measured by the cell Titer-Glo luminescent assay (FIG. 3d-f). Keeping the siRNA concentration fixed (0.5 μM), repeated laser scanning improved knockdown efficiency as well, reaching up to 70% after 4 laser scans, which is similar to a single scan with 5 μM siRNA.

We next investigated the delivery of CRISP-Cas9 ribonucleoproteins (RNPs). After PEN photoporation of H1299 cells with 0.5-4 μM RNPs, cells were allowed to grow for another 48 h before analysis. Exemplary confocal images and flow cytometry histograms are shown in FIG. 3g, h, respectively, confirming successful GFP knockout. GFP knockout efficiency increased along with the RNP concentration (FIG. 3i, j), reaching a knockout efficiency as high as 80% for the highest RNP concentration. Keeping the RNP concentration fixed (0.5 μM), repeated PEN photoporation (N=2, 3, 4) resulted in enhanced knockout efficiency. Together this shows that PEN photoporation is not only able to deliver relatively small biological molecules like siRNA, but also quite large macromolecular complexes like RNPS.

PEN Photoporation Achieves CIRSPR/Cas9 Mediated Gene Knockouts in Human Embryonic Stem Cells without Affecting Cell Functionality.

Next, we turned to human pluripotent stem cells which are relevant for stem cell therapy. Human embryonic stem cells (hESCs) were grown on PEN nanofibers (1% IONP) modified with a Geltrex coating in order to facilitate their attachment and growth. After 3-4 days, the hESCs were PEN photoporated with RD10 (0.5 mg/mL) to investigate delivery efficiency. Quantification of confocal images revealed a gradual increase of delivery efficiency with a concomitant decrease of cell viability, here determined by live/dead staining. When calculating the delivery yield, which is the percentage of living and transfected cells compared to the initial number of cells, a maximum delivery yield of 61% was obtained for I=0.08 J/cm², which further increased to 71% if laser scanning was performed twice (N=2) (FIG. 4a). As a comparison we also delivered RD10 in hESCs by electroporation as an often-used non-viral transfection method for stem cells. With electroporation a delivery yield of only 53% was obtained for the best functioning electroporation program (CE-118) (FIG. 4b). Exemplary confocal images are shown in FIG. 4c of control hESCs as well as PEN photoporated and electroporated hESCs for the most optimal conditions. 24 h post treatment the difference was even more pronounced, with a cell yield of 63% for PEN photoporation and 25% for electroporation (FIG. 4d). This reduction in delivery yield for electroporation was due to a drop in viability from 72% after 2 h to only 34% after 24 h, pointing at long-term adverse effects in electroporated hESCs. To further investigate this, we compared the proliferation of electroporated and PEN photoporated hESCs. PEN photoporated cells were able to immediately recover and grow exponentially just like the untreated cells. Instead, it took the electroporated cells four days to recover and resume exponential growth (FIG. 4e).

Having established that PEN photoporation does not seem to have a great impact on hESC viability and proliferative capacity, next we examined pluripotency transcription factors Oct4 (Pou5f1), Sox2 and Nanog which are crucial for maintaining a pluripotent cell identity. Since we are interested in investigating the effect of the permeabilization method itself, these experiments were performed according to optimized conditions but in the absence of any one or more (macro) molecules otherwise not present in a native T cell. Based on immunostaining and confocal images, PEN photoporated cells did not show any significant differences in comparison with non-treated hESCs (FIG. 4f, g). Furthermore, we differentiation potential of PEN photoporated hESC towards cardiomyocytes was unaltered compared to control cells based on immunostaining of the cardiomyocyte-specific markers TNNT2 and NKX2.5 (FIG. 4h, i). This is expected to be beneficial for downstream applications like differentiation to hESC-derived cardiomyocytes and subsequent transplantation.

Finally, we applied PEN photoporation to the intracellular delivery of CRISPR/Cas9 RNPs in hESCs in order to knockout the IL-2Rgamma (IL-2R) gene on the X chromosome, which is involved in X-linked severe combined immunodeficiency. Sanger sequencing of PEN-photoporated hESCs with 2 μM RNPs revealed a knockout efficiency >60%, demonstrating successful CRISPR/Cas9 mediated gene knockout in difficult to transfect human embryonic stem cells (FIG. 4j, k).

PEN Photoporation Achieves Efficient Gene Knockdown by siRNA Delivery in Primary Human T Cells.

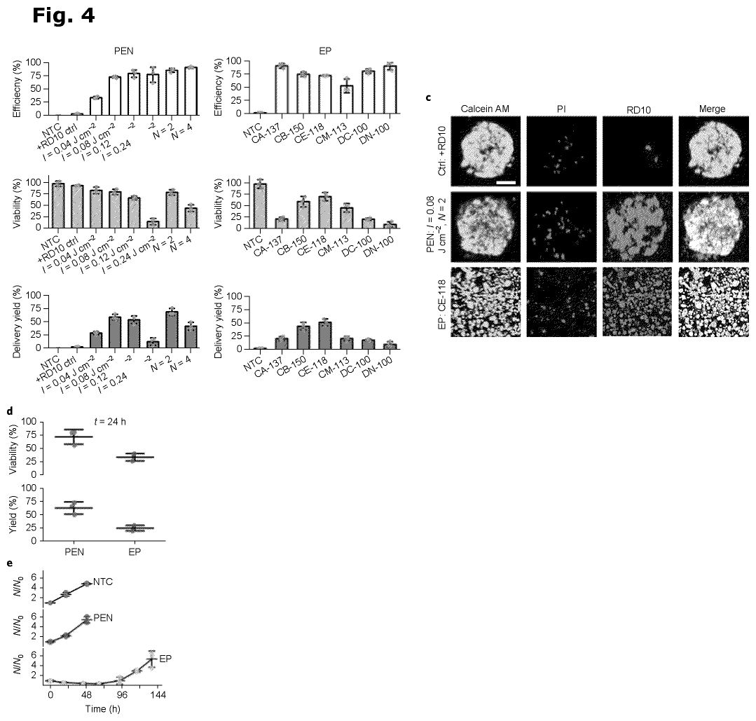

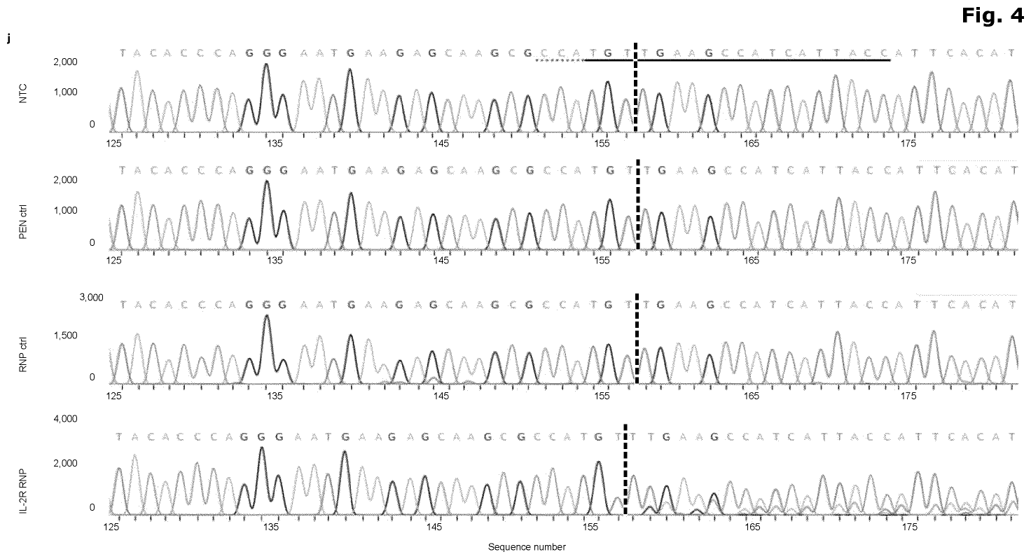

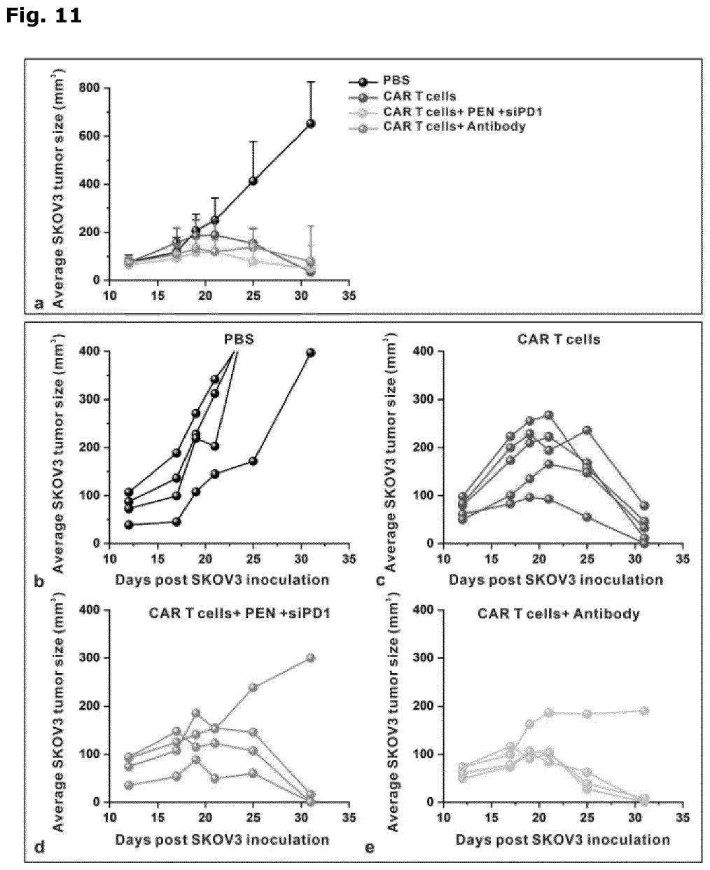

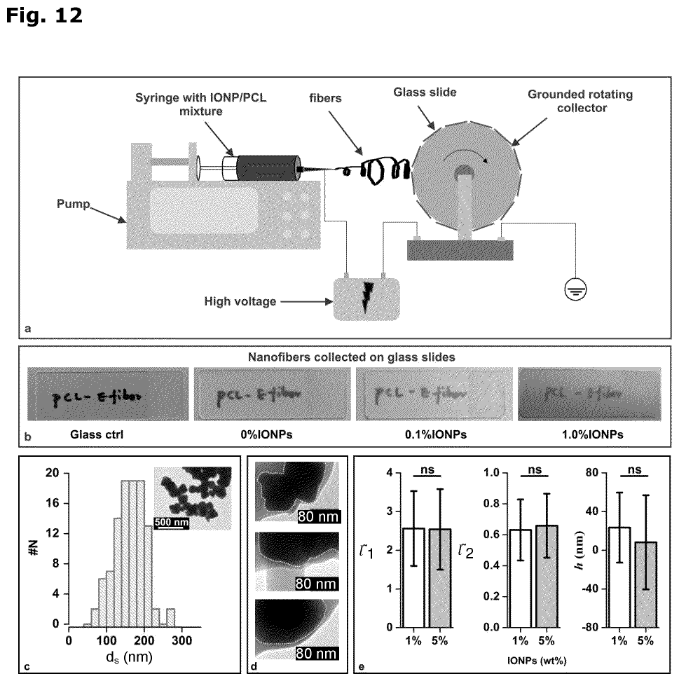

PEN photoporation was applied to human donor-derived T cells (FIG. 13). First, PEN photoporation conditions were optimized by FD10 delivery. Using neutral PEN fibers, an IONP content of 5% was proven optimal with a laser fluence of 0.16 J/cm² (FIG. 14). Using these optimized settings, a direct comparison was made between neutral and hydrated nanofibers which received a treatment with sodium hydroxide to increase their hydrophilicity and enhance cell adhesion. Hydrated nanofibers produced the best results with a yield of 40.7% viable transfected cells with three times laser scanning (FIG. 5a). A comparison was performed with electroporation as the most commonly used non-viral transfection tool for nucleic acid delivery in T cells. Based on the manufacturer's recommendation, several protocols were tried (EO-100, EO-115, FI-115). With a viability of 26.2% and a delivery efficiency of 76.0%, the electroporation protocol EO-100 resulted in the highest FD10 delivery yield (19.3%) (FIG. 5b). While such low cell viability after electroporation may seem surprising considering the manufacturer's claim that >70% T-cell viability is expected for the EO-100 program, it should be noted that this is based on cell viability as measured by live/dead staining and quantification by flow cytometry, which leads to an overestimation of cell viability (FIG. 7).

However, flow cytometry analysis tends to overestimate cell viability as it does not account for fragmented cells which are ‘lost’ in the debris background. Indeed, when we measured the viability of electroporated cells by flow cytometry after Calcein AM staining, an apparently high cell viability of up to 80% was found (FIG. 7a). However, when the same cells were measured by the Cell Titre Glo assay, which measures the remaining metabolic activity of cells compared to the initial population, the viability was found to be much less, reaching 20-30% at best. To confirm that this apparent discrepancy is due to complete cell fragmentation, we determined absolute cell numbers by cell counting before and after electroporation of T cells labeled with Calcium AM (green) and Propidium Iodide (red) (FIG. 7b). Quantification of microscopy images showed that it is in fact more than 30% of cells that are lost by fragmentation after EP (FIG. 7c), both 1 h and 24 h after treatment. The fact that the difference in viabilities between flow cytometry and the Cell Titre Glo assay is even larger than that shows that many of the remaining ‘intact’ cells are actually not very healthy, with reduced metabolic activity even after 24 h of recovery.

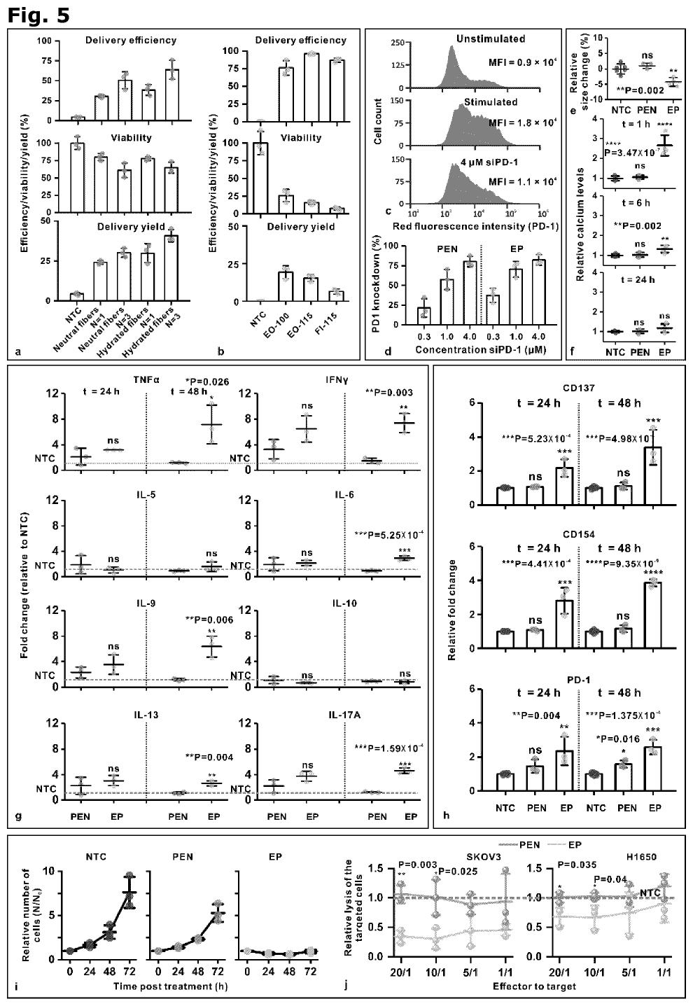

Next, we applied the optimized PEN photoporation and electroporation protocols to deliver siRNA into human T cells to silence expression of the PD1 receptor. PD1 expression is typically upregulated in stimulated T cells and is considered an important mediator of T cell immunosuppression in the tumor micro-environment. Human T cells were cultured for 7 days, transfected by PEN photoporation or electroporation according to the previously optimized conditions and stimulated with CD3/CD28 tetrameric antibody complexes and IL-2 to upregulate PD1 expression. From several tested siRNA constructs (FIG. 8), the D2 siRNA construct was found to perform best (FIG. 9) and was selected for further optimization of PD1 gene silencing. PD1 expression could be silenced in human T cells both by photoporation and electroporation. Silencing became more effective as the siPD1 concentration increased, reaching ˜80% knockdown for 4 μM siPD1 with both PEN photoporation and electroporation (FIG. 5c, d). This shows that PEN photoporation cannot only achieve more living and transfected cells, but also results in a level of downregulation per cell that is similar to electroporation.

PEN Photoporation does not Alter T Cell Homeostasis and Functionality In Vitro, Contrary to Electroporation.

An optimal intracellular delivery technology should minimally disturb the cell's normal functioning and homeostasis, especially when applied to therapeutic cells. Therefore, we compared the downstream effects of PEN photoporation and electroporation on T cell morphology, phenotype and activation state (FIG. 5e-h). Human donor-derived T cells were subjected to PEN photoporation and electroporation in the absence of one or more (macro) molecules otherwise not present in a native T cell so as to investigate the effects induced by the delivery technology itself. First of all, it was noted that electroporated cells had decreased in size 1 h after treatment, which was not the case for photoporated cells (FIG. 5e). This morphological change after electroporation was accompanied by a strong sustained increase in Ca²⁺ levels up to 6 h after treatment, which returned to baseline after 24 h (FIG. 5f). Instead, Ca²⁺ levels remained unaltered for PEN photoporated cells at all times.

Next, we studied production of inflammatory cytokines (TNFα, IFNγ, IL-5, IL-6, IL-9, IL-10, IL-13 and IL-17A) 24 h and 48 h after treatment. In response to PEN photoporation no significant increase of any of the cytokines was observed (FIG. 5g). In contrast, electroporation caused a significant upregulation of most inflammatory cytokines after 48 h (TNFα: 7.2-fold increase, IFNγ: 7.4-fold increase, IL-6:2.9-fold increase, IL-9:6.3-fold increase, IL-13:3.0-fold increase and IL-17A: 4.7-fold increase, compared to non-treated T cells). We continued investigating the extent of upregulation of several activation markers, including CD137 (4-1BB), CD154 (CD40L) and PD-1. All of them were significantly upregulated 24 h and 48 h post electroporation, which was not the case for PEN photoporation except for a slight increase of PD-1 after 48 h (FIG. 5h). Together these results point at phenotypic changes caused by electroporation, which were absent in PEN photoporation-treated T cells.

Next, we validated the functionality of T cells after PEN photoporation and electroporation in vitro. T cell proliferation was investigated first, for which human T cells were PEN photoporated or electroporated (without one or more (macro) molecules otherwise not present in a native T cell), followed by stimulation with CD3/CD28 beads. After electroporation, cell numbers decreased during the first 48 h, but started proliferate again after 72 h. This 2-3 day delay in proliferation post electroporation points to an anergic state (FIG. 5i). Interestingly, PEN photoporation fully preserved the proliferative potential of the human T cells without any significant delay in growth compared to untreated T cells.

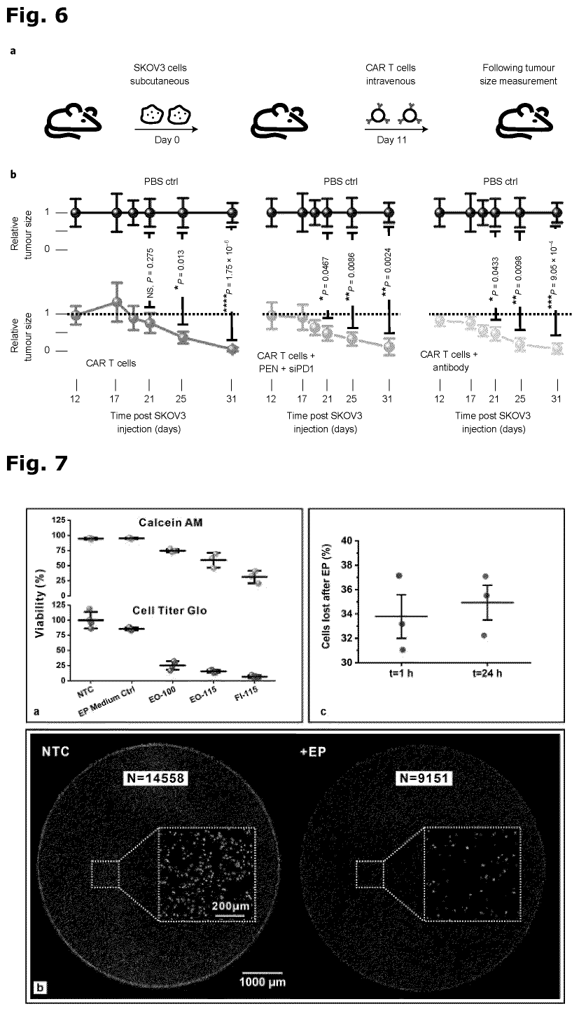

Finally, we compared the cytolytic capacity of electroporated and PEN photoporated T-cells previously transduced with a tumor-targeting chimeric antibody receptor (CAR T cells). The tumor-killing capacity of these CD70-targeted CAR T cells was evaluated in vitro on SKOV3 and H1650 cancer cell lines positive for CD70 antigen and expressing the PD1 ligand (PD-L1) at various levels (FIG. 10). PEN photoporated cells demonstrated efficient tumor cell killing similar to untreated CAR T cells especially for a high effector to target ratio (FIG. 5j). However, electroporation clearly diminished the cytolytic capacity of CAR T cells. Taken together, these results confirm the presence of an anergic state in electroporated cells, which is a consequence of the long-term adverse effects on T cell homeostasis, as was also reported before. In strong contrast, PEN photoporated T cells do not suffer from altered homeostasis and fully retain their cytolytic functionality.

CAR-T Cells Transfected with siPD1 by PEN Photoporation Offer Therapeutic Functionality In Vivo.

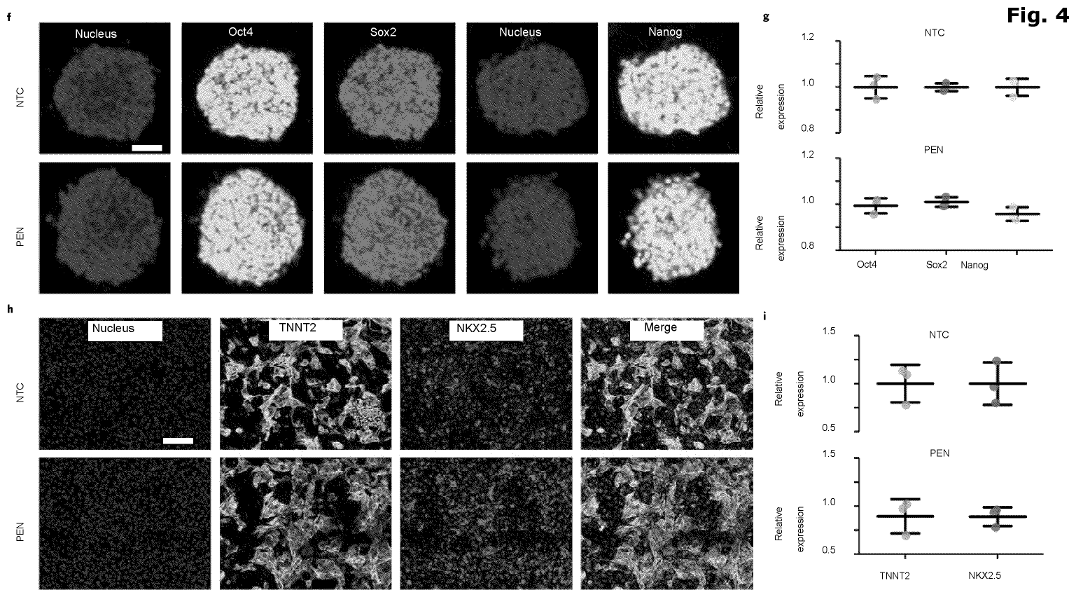

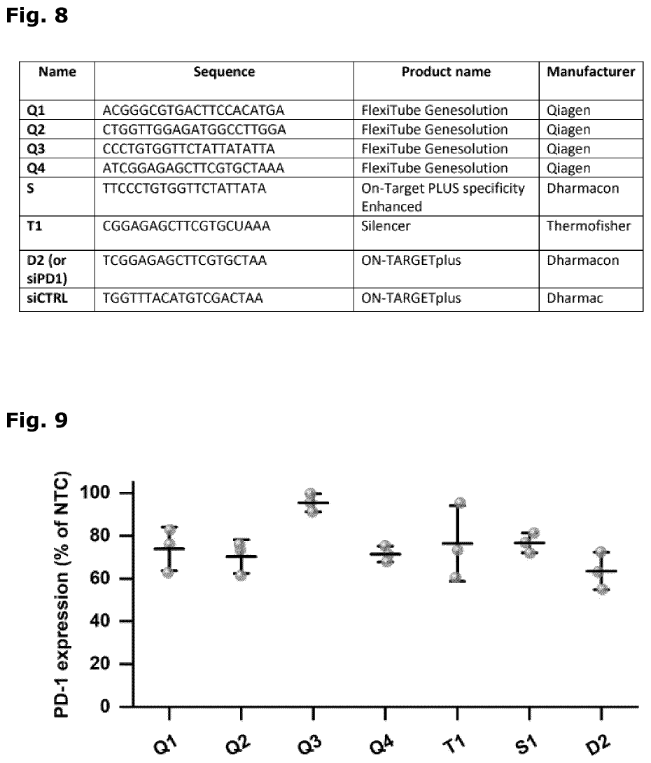

Having confirmed that PEN photoporation does not negatively affect T cell fitness nor the cytolytic potential of CAR T cells, we finally evaluated their efficacy in vivo in a SKOV3 tumor mouse model (FIG. 6a). We found that CAR T cells alone, CAR T cells PEN photoporated with siPD1 and CAR T cells combined with injection of PD1-antibodies can control the tumor growth in a period of one month (FIG. 6b and FIG. 11). Most importantly, we observed that siPD1 treated CAR T cells were able to significantly reduce the tumor volume after already 21 days, which was identical to the positive control with PD-1 antibodies (FIG. 6b). Instead, it took 25 days for CAR T cells alone to significantly control the tumor volume. These in vivo data confirm that PEN photoporated T cells fully retain their therapeutic potential and that siRNA mediated knockdown of the PD-1 receptor can provide a therapeutic advantage for the treatment of solid tumors.

### CONCLUSION

In the abovementioned example the morphology, density and distribution of IONPs embedded in the electrospun nanofibers was characterized. Furthermore, it was shown that both adherent and suspension cells can be safely and efficiently transfected with a range of macromolecules upon irradiation of PEN with nanosecond laser pulses. By performing elemental analysis via inductively coupled plasma-tandem mass spectrometry (ICP-MS/MS), the absence of IONP leakage into the cell medium or cells after laser irradiation was demonstrated.

After demonstrating the possibility to use PEN photoporation to genetically engineer hard-to-transfect cells like embryonic stem cells and human T cells, PEN photoporation is used to transfect CAR-T cells with siPD1, leading to reduced expression of the PD1 receptor and enhancing their tumor killing capacity in vivo. Together it shows that PEN enables cell membrane permeabilization in a variety of cell types without contact to potentially toxic photothermal nanoparticles, thus paving the way towards the use of photoporation for safe and efficient production of gene modified cell therapies.

As a specific example the abovementioned example describes T cells comprising SIRNA, otherwise not present in a native T cell, which siRNA is introduced to the T cells by means of photoporation. Furthermore, the homeostasis of said T cells within 24 h after photoporation is unaffected and comparable to the homeostasis prior to said photoporation or compared to a non-photoporated T-cell. Subsequently, PEN photoporated T cells do not suffer from altered homeostasis and fully retain their cytolytic functionality. Additionally, the example confirms that PEN photoporated T cells fully retain their therapeutic potential and that siRNA mediated knockdown of the PD-1 receptor can provide a therapeutic advantage for the treatment of solid tumors. The example further shows the potential for clinical translation such as for the generation of engineered cells for cell therapies, including adoptive T cell therapy.

It is supposed that the present invention is not restricted to any form of realization described previously and that some modifications can be added to the presented example without reappraisal of the appended claims. For example, the present invention has been described referring to the delivery of siRNA to T cells, but it is clear that the invention can be applied for other macromolecules like for instance other types of nucleic acids, a protein, a peptide, a chemical substance, a polysaccharide, and combinations thereof.

1. A T cell, wherein the T cell is photoporated and wherein the homeostasis of said T cell within at least 24 hours after photoporation is unaffected and comparable to the homeostasis prior to said photoporation or compared to a non-photoporated T-cell.

2. The T cell according to claim 1, wherein one or more macromolecules are introduced in said T cell by means of said photoporation.

3. The T cell according to claim 1, wherein the cell size of said T cell within at least 24 hours after said photoporation differs maximally 3% compared to the cell size of said T cell prior to said photoporation or compared to a non-photoporated T cell.

4. The T cell according to claim 2, wherein the calcium level in said T-cell within at least 24 hours after said photoporation differs maximally 2% compared to the calcium level of said T cell prior to said photoporation or compared to a non-photoporated T cell.

5. The T cell according to claim 1, wherein said photoporation did not result in an upregulation of CD137, PD1 or CD154 within at least 24 hours after said photoporation compared to the levels prior to said photoporation.

6. The T cell according to claim 1, wherein the proliferation N/N₀ of the T cell in a time interval of 0 hours to 72 hours after photoporation increases from at least 1 to at least 2.

7. The T cell according to claim 1, wherein said T cell is a CAR T cell.

8. The T cell according to claim 7, wherein said CAR T cell after said photoporation has maintained a tumour cytolytic capacity that is similar to its non-photoporated counterpart.

9. The T cell according to claim 8, wherein said tumour cytolytic capacity is similar for an effector-to-target ratio of at least 5/1.

10. The T cell according to claim 7, wherein a target of said CAR T cell is at least one of the following targets: CD70, TNFRSF17, ILR3A, SDC1, EGFRvIII, MUC1, FAP, CD44, CD19, MS4A1, CD22, EPCAM, PDCD1, CA9, CD174, TNFRSF8, CD33, CD38, EPHA2, CD274, FOLR1, SLAMF7, CD5, NCAM1, ERBB2, KDR, LICAM, GD2, ULBP1, ULBP2, IL1RAP, GPC3, IL13RA2, ROR1, CEACAM5, MET, EGFR, MSLN, FOLH1, CD23, CD276, CSPG4, CD133, TEM1, GPNMB, PSCA.

11. The T cell according to claim 1, wherein said photoporation occurred by means of photoresponsive organic nanoparticles.

12. The T cell according to claim 11, wherein said photoresponsive organic nanoparticles are embedded in a solid structure.

13. The T cell according to claim 2, wherein said one or more macromolecules are selected from the group consisting of a nucleic acid, a protein, a peptide, a chemical substance, a polysaccharide, and combinations thereof.

14. A population of T cells according to claim 1.

15. A pharmaceutical composition comprising a therapeutically effective amount of T cells according to claim 1 and an excipient.

16.-24. (canceled)

25. A method of treatment comprising administering to a patient in need thereof of the T cell according to claim 1.

26. The method according to claim 25, wherein the administering of the T cell is to a human patient by intravenous, subcutaneous, or transdermal administration.

27. The method according to claim 26, wherein the T cell is allogenic or autologous to the human patient.

28. The method according to claim 27, wherein the human patient has a cell proliferative disease.

29. The method according to claim 28, wherein the cell proliferative disease is an autoimmune disease and wherein the T cell is targeted to autoimmune cells.

30. The method according to claim 28, wherein the cell proliferative disease is a cancer and wherein the T cell is targeted to a cancer-cell antigen.


SEQUENCE LISTING (16 sequences)

SEQ ID NO: 1 - RNA, 19 nt, Artificial Sequence
  other RNA
gguaaugauggcuucaaca

SEQ ID NO: 2 - DNA, 19 bp, Artificial Sequence
  Other DNA
accaccttacagcagcacc

SEQ ID NO: 3 - DNA, 21 bp, Artificial Sequence
  Other DNA
atgatggtcagaaggaggagg

SEQ ID NO: 4 - DNA, 17 bp, Artificial Sequence
  Other DNA
aggacttagcccgtgtc

SEQ ID NO: 5 - RNA, 21 nt, Artificial Sequence
  Other RNA
caagcugacccugaaguucuu

SEQ ID NO: 6 - RNA, 21 nt, Artificial Sequence
  Other RNA
gaacuucagggucagcuuguu

SEQ ID NO: 7 - RNA, 21 nt, Artificial Sequence
  Other RNA
ugcgcuacgaucgacgauguu

SEQ ID NO: 8 - RNA, 21 nt, Artificial Sequence
  Other RNA
caucgucgaucguagcgcauu

SEQ ID NO: 9 - RNA, 21 nt, Artificial Sequence
  Other RNA
acgggcgugacuuccacauga

SEQ ID NO: 10 - RNA, 21 nt, Artificial Sequence
  Other RNA
cugguuggagauggccuugga

SEQ ID NO: 11 - RNA, 21 nt, Artificial Sequence
  Other RNA
cccugugguucuauuauauua

SEQ ID NO: 12 - RNA, 21 nt, Artificial Sequence
  Other RNA
aucggagagcuucgugcuaaa

SEQ ID NO: 13 - RNA, 20 nt, Artificial Sequence
  Other RNA
uucccugugguucuauuaua

SEQ ID NO: 14 - RNA, 18 nt, Artificial Sequence
  Other RNA
cggagagcuucgugcuaa

SEQ ID NO: 15 - RNA, 19 nt, Artificial Sequence
  Other RNA
ucggagagcuucgugcuaa

SEQ ID NO: 16 - RNA, 19 nt, Artificial Sequence
  Other RNA
ugguuuacaugucgacuaa